(19) United States
(12) Patent Application Publication
(10) Pub. No.: US 2004/0121954 A1
(43) Pub. Date: Jun. 24, 2004

(54) Poly(dipeptide) as a drug carrier

(76) Inventor: Wuhan Jingya Xu, Missouri, TX (US)

Correspondence Address: GILBRETH & ASSOCIATES, P.C., PO BOX 2428, BELLAIRE, TX, 77402-2428, US

(21) Appl. No.: 10/659,033

(22) Filed: Sep. 9, 2003

Related U.S. Application Data
(63) Continuation-in-part of application No. 10/104,480, filed on Mar. 20, 2002, now abandoned.
Application No. 10/104,480 is a continuation of application No. 09/291,234, filed on Apr. 13, 1999, now abandoned.

(51) Int. Cl. A61K 38/17; A61K 31/337

(52) U.S. Cl. 514/12; 514/449; 530/350

(57) ABSTRACT

A novel polypeptide drug carrier is provided wherein polypeptides containing glutamic acid and aspartic acid, or glutamic acid/alanine, or glutamic acid/asparagine, or glutamic acid/glutamine, or glutamic acid/glycine, are conjugated to drugs in order to improve the solubility of the drugs and/or their therapeutic efficacy in vivo. An illustrative example involves the conjugation of paclitaxel to a poly(glutamic acid/aspartic acid) polypeptide and its efficacy in the treatment of prostate cancer in vivo.

### RELATED-APPLICATION DATA

This patent application is a Continuation-in-part of copending U.S. patent application Ser. No. 10/104,480, filed Mar. 20, 2002, which is a Continuation of U.S. patent application Ser. No. 09/291,234, filed Apr. 13, 1999, both of which are incorporated herein by reference.

### BACKGROUND OF THE INVENTION

1. Field of the Invention

The present invention relates to novel drug carriers and their use. More particularly, the present invention relates to the novel use of a poly(di-peptide) peptide covalently bound to a drug to act as a drug carrier, particularly for poorly water soluble drugs. The poly(dipeptide) may be composed of a combination of glutamic acid and aspartic acid. The poly(dipeptide) may be composed of combinations of glutamic acid with alanine, asparagine, glycine or glutamine.

2. Description of the Related Art

It is undisputed that advances in pharmaceuticals have revolutionized health care for humans and other animals as well. However, despite the outstanding advances made in the field of pharmacology, some significant limitations still remain in the treatment of various diseases via drug agents. One of the most significant limitations at this time relates to the delivery of particular drugs in vivo, especially in situations where drugs are poorly water soluble. Indeed, the use of some drugs which show great promise in vitro, has been severely limited due to issues related to their solubility. This causes problems with drug delivery in vivo. One example of such a drug is paclitaxel in the treatment of tumors, especially for example, in the case of prostate cancer.

As discussed below, the prior art has attempted to address this issue in a number of ways. However, as presented in more detail below, prior to the instant invention, the unique advantages of conjugating a drug to the inventive polypeptide, while desired, were unknown.

U.S. Pat. No. 4,675,381, issued to Bichon, on Jun. 23, 1987, entitled “Biodegradable Polypeptide and its Use for the Gradual Release of Drugs,” discloses a polyaspartate and/or polyglutamate polymer as a drug carrier. This patent envisions the use of polyaspartate and/or polyglutamate polymers as drug carriers wherein the drug is encapsulated or incorporated in the matrix of the polymer. The patent does not disclose, teach or suggest covalent conjugates of the drug with the polymer. Furthermore, most of the teaching in the patent is directed to homopolymers of aspartate and glutamate, not combinations of the two amino acids.

U.S. Pat. No. 5,087,616 issued to Myers et al. on Feb. 11, 1992, entitled “Cytotoxic Drug Conjugates and Their Delivery to Tumor Cells,” discloses the use of a biodegradable polymeric carrier to which one or more cytotoxic molecules, for instance, daunomycin is conjugated. The biodegradable polymeric carrier is specified to be, for example, a homopolymer of polyglutamic acid. However, the use of a drug conjugated to a di-peptide copolymer carrier is clearly not disclosed, taught or suggested in this reference.

A 1983 J. Med. Chem. paper by Piper et al. entitled “A Synthetic Approach to Poly(γ-glutamyl) Conjugates of Methotrexate” discloses the use of methotrexate conjugated to 2 to 3 glutamic acid units. This paper does not disclose, teach or suggest di-peptide polymers of glutamic acid and aspartic acid, or glutamic acid with alanine, asparagine, glutamine or glycine.

A 1982 Int. J. Cancer paper by Zunino et al. entitled “Anti-Tumor Activity of Daunorubicin Linked to Poly-L-Aspartic Acid” discloses daunorubicin bound to a homopolymer of polyaspartic acid. The paper indicates that “the binding (of daunorubicin) to the polypeptide markedly reduced drug toxicity but only slightly decreased drug potency.” “The daunorubicin-poly-L-aspartic acid conjugate demonstrated anti-tumor activity comparable to that of doxorubicin in leukemia models, but superior to that of doxorubicin in a solid tumor model.” While this paper does disclose the covalent conjugation of an anti-tumor drug to a homopolymer of polyaspartic acid, it does not disclose, teach or suggest the use of a di-peptide containing polymer of aspartic acid and glutamic acid, or glutamic acid with alanine, asparagine, glutamine or glycine.

A 1998 Cancer Research paper by Li et al. entitled “Complete Regression of Well-established Tumors Using a Novel Water-soluble Poly(L-Glutamic Acid)-Paclitaxel Conjugate,” discloses the use of a water-soluble poly-L-glutamic acid-paclitaxel conjugate to produce tumor effects with diminished toxicity. However, this paper does not disclose, teach or suggest the use of the inventive copolymer.

A 1989 J. Pharm. Exp. Ther. paper by Ramsammy entitled “Polyaspartic Acid Protects Against Gentamicin Nephrotoxicity in the Rat,” discloses the use of poly-amino acids, including polyaspartic acid, to provide protection against the development of amino glycoside-induced nephrotoxicity in the rat. However, this paper does not disclose, teach or suggest the inventive copolymer, much less the inventive copolymer conjugated to a drug.

A 1990 Biopolymers paper by Hayashi and Iwatsuki, entitled “Biodegradation of Copoly(L-Aspartic Acid/L-Glutamic Acid) In Vitro,” discloses the preparation of copolypeptides consisting of L-aspartic acid and L-glutamic acid. The paper describes the use of such polypeptides to determine the effects of copolymer composition and sequential distributions on the rate of degradation by papain to stimulate in vivo polymer degradation. This paper does disclose, teach or suggest the use of copolymers of glutamic acid and aspartic acid, similar to the copolymer of the present invention. The paper also does not disclose, teach or suggest the use of such copolymers covalently conjugated with drugs.

U.S. Pat. No. 4,960,790 issued to Stella et al., and entitled “Derivatives of Taxol, Pharmaceutical Compositions Thereof and Methods for the Preparation Thereof” discloses the anti-tumor agent taxol covalently conjugated with, for example, an amino acid (for example, glutamic acid). However, this patent does not disclose, teach or suggest the use of large polypeptides, much less the use of the inventive polypeptide containing glutamic acid and aspartic acid, or glutamic acid with alanine, asparagine, glutamine or glycine.

Finally, a 1960 J. Am. Chem. Soc. by Karlson et al., entitled “The Helical Sense of Poly-β-benzyl-L-aspartate” discusses the physical characteristics of series of copolymers derived from γ-benzyl-L-glutamate and β-benzyl-L-aspartate. However, this paper does not disclose, teach or suggest the use of such copolymers in vivo much less their conjugation with anti-tumor drugs.

As indicated from the above art, there has been a long-felt need in the art to attempt to solubilize poorly soluble drugs, such as anti-tumor agents, and in this endeavor, the art has attempted to accomplish this by various means, including the use of, for example, polypeptides comprising homopolymers of poly-glutamic acid and poly-aspartic acid. However, as discussed in more detail below, the present inventive di-peptides conjugated to drugs to increase their solubility in vivo, while desired in the art, has not been anticipated or suggested by the art.

In order to demonstrate one embodiment of the present invention, a conjugate of the antitumor agent paclitaxel was made with the inventive polypeptide (for example, a poly(dipeptide) comprising glutamic acid and aspartic acid) and used as a drug delivery vehicle. It was then shown that this inventive conjugate possessed superior biological and therapeutic properties in vivo over, for example, unconjugated drug, and the drug conjugated with known prior art carriers (e.g., homopolymers of glutamic acid and aspartic acid). Data below shows that, for example, conjugating the antitumor drug paclitaxel to the inventive polymer—and only the inventive polymer—results in unexpected therapeutic properties of paclitaxel such as the treatment of prostate cancer. Indeed, the following results show what applicants believe is the first described efficacy of paclitaxel in any form against prostate tumors in vivo.

Paclitaxel was selected for an exemplary embodiment of a drug to be conjugated with the inventive carrier as an example of the practice of the instant invention because paclitaxel is a known antitumor drug, with known solubility problems in vivo. Hence, it has known effectiveness problems and toxicity problems in vivo related to its stability and related in vivo use. Furthermore, the need for the present invention (i.e., a carrier that can solubilize and/or enhance the in vivo therapeutic use of drugs, for example, poorly soluble drugs, such as, for example, poorly soluble antitumor drugs) is amply demonstrated by paclitaxel since the prior art has made a number of attempts at conjugating the drug to various carriers, including polypeptides, in order to improve the biological use (see above, Background of the Invention).

Paxlitexel is an antitumor agent that works as an antitumin agent. Improvements of cancer treatment is extensively determined by the development of more tumor specific pharmaceuticals and new drug techniques. Due to an angiogenesis process involved in the tumor vascular density, antitubular agents have opened a new era in the treatment of various tumors and have undergone extensive preclinical development and evaluation.

Tubulin is a principal protein of subunit of microtubulars. Microtubulars assemble when they are required by a cell for a particular function and depolymerize when they are no longer needed. Therefore, tubulin is a cellular target for antimitotic agents. Some of these agents, such as vincristine, vinblastine, rhizoxin, maytansin and epipodophyllotoxins interact with tubulin on the colchicine binding sites to inhibit tubular polymerization and thereby cause cellar rest metephase. Paclitaxel on the other hand, acts to promote assembly of micotubulars resulting in highly stable but non-functional polymers that lead to mitotic for rest of poliferating cells. Schiff, P. B., Horwitz, S. B. Proc. Natl. Acad. Sci. USA. 1980, 77, 1561-1565; Schiff, P. B., Fant, J., Horwitz, S. B. Nature (London) 1980, 283, 665-667; Rowinsky, E. K., Cazenave, L. A., Donehower, R. C. JCNI, J. Natl Cancer Inst. 1990, 82, 1247-1259, Imbert, T. F. Biochimie, 1998, 80, 207-222; Sandler, E. S., Friedman, D. J., Mustafa, M. M., Winick, N. J., Bowman, W. P., Buchanan, G. R. Cancer 1997 79(5), 1049-1054.

In the art, paclitaxel formulated in the cremophor has been used to treat breast, ovarian, colon and lung cancers (Rowinsky, E. K.; Donehower, R. C. Cancer Res. 1998, 58, 2404-2409; Holmes, F. A., Kudelka, A. P., Kavanagh, J. J., Huber, M. H., Ajani, J. A., Valero, V. In: G. I. Georg. T. T Chen I. Ojima and D. M Vyas (eds). 1995,31-57; Cortes, J. E.; Padur,, R. J., Clin. Oncology. 1995, 13(10), 2643-2655). However, despite some effectiveness of paclitaxel, there are notable side effects such as granulocytopenia and body weight loss (Rowinsky, E. K. and Donehower, R. C., Review: Paclitaxel (Taxol), New Eng. J. Med. 1995, 332, 1004-1014). It is well known in the art that the poor water solubility of paclitaxel makes the drug difficult to administer intraveneously.

Furthermore, and importantly, it is known in the art that paclitaxel, while showing some effectiveness in the breast and ovarian cancers, is not effective in the treatment of prostate cancer.

Paclitaxel was, therefore, chosen, as a exemplary drug in which to conjugate to the inventive di-glutamic acid/aspartic acid polypeptide in order to determine whether conjugation in the inventive complex produces a drug carrier complex which shows improved therapeutic use. As discussed below, this is indeed the case, and only when paclitaxel is conjugated to the inventive conjugate has it ever been shown to be effective in vivo against prostate cancer.

### SUMMARY OF THE INVENTION

An object of the invention is the provision of a therapeutic compound including a drug carrier.

An object of the invention additionally is a method for improving the solubility of a drug moiety.

An additional object of the invention is a method for treating a condition.

Thus, in accomplishing the foregoing objectives, there is provided in accordance with one aspect of the present invention a therapeutic compound comprising at least one drug moiety, and at least one polypeptide drug carrier moiety, the drug moiety being covalently linked to the carrier moiety, and the polypeptide drug carrier moiety comprising glutamic acid and a second amino acid selected from the group consisting of aspartic acid, alanine, asparagine, glutamine, glycine, and combinations of two or more amino acids selected from the group consisting of aspartic acid, alanine, asparagine, glutamine, and glycine.

In a specific embodiment, the second amino acid is aspartic acid, the drug moiety is selected from the group consisting of anti-tumor drugs, cardiovascular drugs, anti-microbial drugs, diabetic drugs, anti-inflammatory drugs, and pain alleviating drugs.

In a specific embodiment, the drug moiety is selected from the group of drugs consisting of, for example, paclitaxel, epipodophyllotoxin, podophyllotoxin, vincristine, docetaxel, daunomycin, doxorubicin, mitoxantrone, topotecan, bleomycin, gemcitabine, fludarabine and 5-FUDR.

In a preferred embodiment, the drug moiety is paclitaxel.

In a specific embodiment, the polypeptide drug carrier moiety comprises from about 50 to about 90 percent, by weight, glutamic acid, and from about 10 to about 50 percent, by weight, aspartic acid, or alanine, or asparagine, or glutamine, or glycine, or combinations thereof, more preferably from about 60 to about 80 percent, by weight, glutamic acid, and from about 20 to about 40 percent, by weight, aspartic acid, or alanine, or asparagine, or glutamine, or glycine, or combinations thereof, and most preferably from about 70 to about 75 percent, by weight, glutamic acid, and from about 25 to about 30 percent, by weight, aspartic acid, or alanine, or asparagine, or glutamine, or glycine, or combinations thereof.

In another embodiment, the therapeutic compound comprises at least two drug moieties, which may not the same as each other.

In another embodiment, the therapeutic compound comprises a plurality of drug moieties.

In still another embodiment, the drug moiety of the therapeutic compound comprises from about 10 percent to about 60 percent, by weight, more preferably from about 20 percent to about 50 percent, by weight, and most preferably from about 20 percent to about 40 percent, by weight of the therapeutic compound. Moreover, the polypeptide drug carrier moiety may comprise from about 40 percent to about 90 percent, by weight, more preferably from about 50 percent to about 80 percent, by weight, and most preferably from about 60 percent to about 80 percent, by weight of the therapeutic compound.

In preferred embodiments (for example, paclitaxel with a poly(dipeptide) glutamic acid/aspartic acid carrier), the drug moiety does not comprise more than about 60% by weight of the therapeutic compound (in order to not adversely affect solubility and/or viscosity which can effect injectability of the compound).

In a preferred embodiment, the drug moiety is paclitaxel, the carrier moiety comprises about 70 percent glutamic acid and about 30 percent aspartic acid, the paclitaxel drug moiety is about 20 percent to about 40 percent, by weight, of the therapeutic compound, and the molecular weight of the therapeutic compound is from about 20,000 to about 50,000 daltons.

Further in accomplishing the foregoing objectives, there is provided in accordance with another aspect of the present invention a method for improving the solubility of a drug moiety comprising the steps of covalently conjugating at least one drug moiety with at least one polypeptide drug carrier moiety, thereby creating a therapeutic compound, the therapeutic compound comprising at least one drug moiety, and at least one polypeptide drug carrier moiety, the drug moiety being covalently linked to the carrier moiety, and the polypeptide drug carrier moiety comprising glutamic acid and aspartic acid or alanine, or asparagine, or glutamine, or glycine, or combinations thereof.

In a preferred embodiment, the water solubility of the therapeutic compound is greater than the water solubility of the drug moiety.

In another embodiment, the drug moiety is an antitumor drug.

In a preferred embodiment, the drug moiety is paclitaxel.

In a specific embodiment, the polypeptide drug carrier moiety comprises from about 50 to about 90 percent, by weight, glutamic acid, more preferably from about 60 to about 80 percent, by weight, glutamic acid, and most preferably from about 70 to about 75 percent, by weight, glutamic acid, and from about 10 to about 50 percent, by weight, aspartic acid, or alanine, or asparagine, or glutamine, or glycine or combinations thereof, more preferably from about 20 to about 40 percent, by weight, aspartic acid, or alanine, or asparagine, or glutamine, or glycine, or combinations thereof, and most preferably from about 25 to about 30 percent, by weight, aspartic acid, or alanine, or asparagine, or glutamine, or glycine, or combinations thereof.

Further regarding the role of alanine, asparagine, glutamine, and/or glycine in the present invention, the following is noted. It is believed at the time of the application that a preferred embodiment uses a poly(dipeptide) polymer of glutamic acid and aspartic acid. However, it is also believed, but not in any limiting sense, that any amino acids similar to aspartic acid, including alanine, asparagine, glutamine, and glycine, can be substituted for aspartic acid in the inventive poly(dipeptide). While not wishing to be bound in anyway, at the time of the application it is believed that a key aspect of the inventive poly(dipeptide) relates to the glutamic acid backbone. It is believed that as long as glutamic acid is present in the poly(dipeptide), aspartic acid may serve as the other amino acid, or any amino acid similar to aspartic acid, such as, for example, alanine, asparagine, glutamine, and glycine may be used. These amino acids may be substituted in whole or in part for aspartic acid and may be mixed. Giving a plurality of inventive poly(dipeptide) polymers, each having glutamic acid, and otherwise containing aspartic acid, or alanine, or asparagine, or glutamine, or glycine or any combinations thereof.

Further in accomplishing the foregoing objectives, there is provided in accordance with another aspect of the present invention a method for treating a condition comprising the steps of administering a therapeutically effective amount of a therapeutic compound comprising at least one drug moiety, and at least one polypeptide drug carrier moiety, the drug moiety being covalently linked to the carrier moiety, and the polypeptide drug carrier moiety comprising glutamic acid and a second amino acid selected from the group consisting of aspartic acid, alanine, asparagine, glutamine, glycine, and combinations of aspartic acid, alanine, asparagine, glutamine, and glycine.

In a specific embodiment, the drug moiety is selected from the group consisting of anti-tumor drugs, including, for example, paclitaxel, epipodophyllotoxin, vincristine, docetaxel, daunomycin, doxorubicin, mitoxantrone, topotecan, bleomycin, gemcitabine, fludarabine and 5-FUDR.

In an embodiment, the polypeptide drug carrier moiety comprises from about 50 to about 90 percent, by weight, glutamic acid, more preferably from about 60 to about 80 percent, by weight, glutamic acid, and most preferably from about 70 to about 75 percent, by weight, glutamic acid, and from about 10 to about 50 percent, by weight, aspartic acid, or alanine, or asparagine, or glutamine, or glycine, or combinations thereof, more preferably from about 20 to about 40 percent, by weight, aspartic acid, or alanine, or asparagine, or glutamine, or glycine, or combinations thereof, and most preferably from about 25 to about 30 percent, by weight, aspartic acid, or alanine, or asparagine, or glutamine, or glycine, or combinations thereof.

In a preferred embodiment, the condition is a prostate tumor and the therapeutic agent is paclitaxel.

Further still, in summary, the present invention relates to the discovery that a particular polypeptide composed of glutamate and aspartate makes an unexpectedly good carrier for delivery of drugs, including poorly soluble drugs. An illustrative example includes anti-tumor agents. More particularly, and for example, the present invention relates to the synthesis and use of a poly (glutamate/aspartate) peptide of approximately 26,000-30,000 molecular weight, containing approximately 70% glutamic acid and 30% aspartic acid, covalently linked with a drug. Such drug may be, for example, a poorly soluble drug and/or an anti-tumor agent. One example of a preferred embodiment is the conjugation of the anti-tumor drug paclitaxel. In a preferred embodiment, the concentration of the conjugated drug, for example, paclitaxel, may be from approximately 20% to 40% by weight of the overall conjugate.

As described in more detail below, the present inventors have discovered that the use of the instant inventive conjugate (poly-glutamate/aspartate polypeptide and poly-glutamate/alanine, asparagine, glutamine, glycine) results in unexpectedly good in vivo properties when covalently linked to drugs. These properties are superior to that found for the conjugation of drugs to other drug carriers known in the art, such as other polypeptides, including homopolymers of glutamic acid and aspartic acid. In particular, and for example, one use of the instant invention involves the conjugation of paclitaxel to the inventive peptide to enable the effective in vivo treatment of prostate cancer. As shown in more detail below, conjugation of paclitaxel known prior art polypeptide carriers, or use of unconjugated paclitaxel, results in ineffective treatment of prostate cancer in vivo. However, conjugation of paclitaxel to the unique inventive copolymer results in the first ever observed therapeutic treatment of prostate cancer in animals.

As further embodiments of any or all of the above embodiments, the polypeptide drug carrier moiety comprises glutamic acid and a second amino acid, with at least one of the glutamic acids bonded directly to at least one second amino acid, preferably bonded directly to two second amino acids. Preferably, at least 10% of the glutamic acids, more preferably at least 20%, even more preferably at least 50%, still more preferably at least 60%, and yet more preferably at least 75% of the glutamic acids are bonded directly to at least one acid.

### BRIEF DESCRIPTION OF THE DRAWINGS

FIG. 1A shows a schematic of the synthesis of the inventive polypeptide poly(glutamic/aspartic acid).

FIG. 1B shows a sample amino acid analysis of a sample of the inventive poly(glutamic/aspartic acid) di-peptide.

FIG. 2A shows a synthetic scheme conjugating paclitaxel to poly(glutamate/aspartate).

FIG. 2B shows an NMR spectra of the inventive copolymer poly(glutamate/aspartate).

FIG. 2C shows an NMR spectra of unconjugated paclitaxel.

FIG. 2D shows NMR specra of an inventive conjugate of paclitaxel-poly(glutamate/aspartate).

FIG. 3A shows a UV scan of unconjugated paclitaxel.

FIG. 3B shows a UV scan of paclitaxel in the inventive conjugate (i.e., paclitaxel conjugated to poly(glutamate/aspartate)).

FIG. 4A shows a UV scan of the inventive polypeptide conjugated to paclitaxel (paclitaxel-poly(glutamate/aspartate)).

FIG. 4B shows a UV scan of the inventive polypeptide (poly(glutamate/aspartate)) in unconjugated form.

FIG. 5A shows a UV scan standard curve for unconjugated paclitaxel.

FIG. 5B shows a UV scan of paclitaxel conjugated to poly(glutamate/aspartate).

FIG. 6 shows an HPLC analysis of paclitaxel.

FIG. 7 shows an HPLC analysis of a sample paclitaxel-poly(glutamate/aspartate).

FIG. 8 shows an HPLC analysis of an unconjugated inventive di-peptide (poly(glutamate/aspartate)).

FIG. 9A shows an HPLC chromatogram of a mixture of a poly(glutamate/aspartate)-paclitaxel conjugate and unconjugated paclitaxel.

FIG. 9B shows an HPLC 3D chromatogram of a paclitaxel-poly(glutamate/aspartate) polypeptide.

FIG. 9C shows an HPLC 3D chromatogram of a mixture of unconjugated paclitaxel and the inventive paclitaxel-poly(glutamate/aspartate acid) polypeptide conjugate.

FIG. 9D shows the sustained release properties of a poly(glutamate/aspartate) acid-paclitaxel conjugate.

FIG. 9E shows a useful-life determination of a paclitaxel-poly(glutamic/aspartic acid) conjugate.

FIG. 9F shows a in vitro cell culture assay of paclitaxel and the conjugate.

FIG. 9G shows cytotoxicity (IC-50) of the conjugate and of paclitaxel on human prostate cancer cells (PC3) in vitro.

FIG. 10 shows in vivo antitumor activity of an inventive paclitaxel-poly(glutamate/aspartate) conjugate compared to unconjugated paclitaxel against mice bearing ovarian tumor.

FIG. 11 shows in vivo antitumor activity of paclitaxel-polyglutamic acid (homopolymer) and unconjugated paclitaxel in vivo in mice against ovarian tumor.

FIG. 12 shows in vivo antitumor activity of paclitaxel conjugated with poly(glutamic acid) and unconjugated paclitaxel in nude mice bearing human breast cancer.

FIG. 13A shows the in vivo antitumor activity of an inventive paclitaxel-poly(glutamic acid/aspartic acid) polypeptide and unconjugated paclitaxel in nude mice bearing human prostate cancer.

FIG. 13B shows the in vivo antitumor activity of paclitaxel conjugated to polyglutamic acid homopolymer and unconjugated paclitaxel in nude mice bearing human prostate cancer.

FIG. 14 shows in vivo antitumor activity of paclitaxel-poly(glutamic acid/aspartic acid) conjugate, paclitaxel-poly glutamate (homopolymer) conjugate, paclitaxel-polyaspartic (homopolymer) conjugate and unconjugated paclitaxel in nude mice bearing human prostate cancer.

FIG. 15A shows the antitumor activity of paclitaxel conjugated to the inventive di-peptide poly(glutamic acid/aspartic acid) compared to unconjugated taxol in breast tumor-bearing athymic nude mice at 15 days post treatment.

FIG. 15B shows antitumor activity of poly(glutamic acid/aspartic acid) conjugated to paclitaxel, compared to unconjugated paclitaxel in nude mice bearing human breast tumor at 43 days post treatment.

FIG. 16 shows antitumor activity of paclitaxel conjugated to poly(aspartic acid/glutamic acid) compared with unconjugated paxol in prostate tumor-bearing athymic nude mice at 15 day post treatment.

FIG. 17A shows antitumor activity of paclitaxel conjugated with poly(glutamnic acid/aspartic acid) compared with unconjugated paclitaxel and paclitaxel conjugated with polyglutamic acid homopolymer on nude mice bearing human prostate tumor (mice at 48 hours post treatment).

FIG. 17B shows antitumor activity of paclitaxel conjugated with poly(glutamic acid/aspartic acid) compared with unconjugated paclitaxel and paclitaxel conjugated with polyglutamic acid homopolymer on nude mice bearing human prostate tumor (mice at 7 days post-treatment).

FIG. 17C shows antitumor activity of paclitaxel conjugated with poly(glutamic acid/aspartic acid) compared with unconjugated paclitaxel and paclitaxel conjugated with polyglutamic acid homopolymer on nude mice bearing human prostate tumor (mice at 22 days post-treatment).

FIG. 18, shows an amino acid analysis of the poly(glutamic acid/aspartic acid) of Example 20.

FIG. 19A shows the structures of etoposide.

FIG. 19B shows the structures of podophyllotoxin.

FIGS. 20A and 20B are graphs of data showing a comparison between poly(dipeptide)-podophyllotoxin and etoposide for human prostate cancer cells (FIG. 20A) and human breast cancer cells (FIG. 20B).

The drawings are not necessarily to scale, and certain features of the invention may be exaggerated in scale or shown in schematic form in the interest of clarity and conciseness.

### DETAILED DESCRIPTION OF THE INVENTION

It is readily apparent to one skilled in the art that various substitutions and modifications may be made to the invention disclosed herein without departing from the scope and spirit of the invention.

The term “therapeutic” as used here, for example, in the terms “therapeutic compound” and “therapeutically effective amount” means to have at least some minimal physiological effect. For example, a “therapeutic compound” would have at least some minimal physiological effect upon being administered to a living body. An agent may have at least some minimal physiological effect upon administration to a living body if, for example, its presence results in a change in the physiology of a recipient animal. For example, a physiological effect upon administering a “therapeutic” anti-tumor compound may be the inhibition of tumor growth, or decrease in tumor size, or prevention reoccurrence of the tumor. Administration of a “therapeutically effective amount” means the amount administered is physiologically significant. An agent is physiologically significant if its presence results in a change in the physiology of a recipient animal. For example, in the treatment of cancer or neoplastic disease, a compound which inhibits the growth of a tumor or decreased the size of the tumor or prevents the reoccurrence of the tumor would be considered therapeutically effective.

The term “anti-tumor drug” means any therapeutic agent having therapeutic effect against a tumor, neoplastic disease or cancer.

The term “drug” means any agent having a therapeutic effect when administered to an animal.

The dosage of the present administration for therapeutic treatment will be sufficient to generate a therapeutically effective amount of the administered agent.

The term “condition” means any condition, state, disease, abnormality, imbalance, malady and the like in an animal which one seeks to effect by administrating a therapeutically effective amount of a therapeutic compound. A condition may include, but is not limited to, cancers, neoplastic diseases, tumors, and conditions of the prostate, including prostate tumors and/or prostate cancer.

The term “treating”, used for example in the term “treating a condition”, means at least the administration of a therapeutically effective amount of a therapeutic compound to elicit a therapeutic effect. It does not necessarily imply “curing”, but rather having at least some minimal physiological effect upon a condition upon administration to a living body having a condition. For example, treatment could encompass administering an agent and the presence of that agent resulting in a change in the physiology of a recipient animal.

The terms “peptide”, “polypeptide”, “di-peptide”, “copolymer”, “poly(glutamic acid/aspartic acid)” (and all variations thereupon), and “inventive peptide” can refer to the peptide of the present invention as further defined herein (and comprising, for example, a polypeptide comprising aspartic acid and glutamic acid and/or polypeptides comprising aspartic acid with alanine, asparagine, glutamine and glycine, in any combination).

Dosage and Formulation

The therapeutic compounds (including compounds, drugs, conjugates and the like) of this invention can be formulated and administered to treat a variety of conditions. They can be administered by any conventional means available for use in conjunction with pharmaceuticals, either as individual therapeutic active ingredients or in a combination of therapeutic active ingredients. They can be administered alone, or with a pharmaceutical carrier selected on the basis of the chosen route of administration and standard pharmaceutical practice.

The dosages are determined for the chosen therapeutic use, including the condition to be treated, the therapeutic agent used to treat the condition and the type of animal treated (including considerations as to age, weight, sex and so forth). Such determinations are well within the scope of those skilled in the art and do not involve undue experimentation or exercise of inventive skill.

The dosage administered will be a therapeutically effective amount of active ingredient and will, of course, vary depending upon known factors such as a the pharmacodynamic characteristics of the particular active ingredient and its mode and route of administration; age, sex, health and weight of the recipient; nature and extent of symptoms; kind of concurrent treatment, frequency of treatment and the effect desired. Usually a daily dosage (therapeutic effective amount) of active ingredient can be about 1 to 400 milligrams per kilogram of body weight. Ordinarily, 1 to 200, and preferably 1 to 50, milligram per kilogram per day given in dividend doses 2 to 4 times a day or in sustained release form is effective to obtain desired results.

Dosage forms (compositions) suitable for internal administration contain from about 1.0 to about 500 milligrams of active ingredient per unit. In these pharmaceutical compositions, the active ingredient will ordinarily be present in an amount of about 0.05-95% by weight based on the total weight of the composition.

Administration may be by any means suitable for the condition to be treated and may include, for example, oral administration. Such determination is within the ordinary level of skill of one skilled in the art. For example, oral administration may be accomplished using solid dosage forms such as capsules, tablets and powders, or in liquid dosage forms such as elixirs, syrups, emulsions and suspensions. The therapeutic compound (agent or the like) may also be, for example, parenterally administered by injection, rapid infusion, nasopharyngeal adsorption of dermoabsorption. The agent may also be administered intramuscularly, intravenously, or as a suppository.

Gelatin capsules may contain the therapeutic compound and powdered carriers such as lactose, sucrose, mannitol, starch, cellulose derivatives, magnesium stearate, stearic acid, and the like. Similar diluents can be used to make compressed tablets. Both tablets and capsules can be manufactured as sustained release products to provide for continuous release of medication over a period of hours. Compressed tablets can be sugar coated or film coated to mask any unpleasant taste and protect the tablet from the atmosphere, or enteric coated for selective disintegration in the gastrointestional tract.

Liquid dosage forms for oral administration can contain coloring and flavoring to increase patient acceptance.

In general, water, a suitable oil, saline, aqueous dextrose (glucose), and related sugar solutions and glycols are suitable carriers for parenteral solutions. Solutions for parenteral administration may contain a water soluble salt of the therapeutic compound (agent and the like), suitable stabilizing agents and, if necessary, buffer substances. Antioxidizing agents such as sodium bisulfate, sodium sulfite or ascorbic acid either alone or combined are suitable stabilizing agents. Also used are citric acid and its salts and sodium EDTA. In addition, parenteral solutions can contain preservatives such as benzalkonium chloride, methyl- or propyl-prarben and chlorobutanol. Suitable pharmaceutical carriers are descried in Remington's Pharmaceutical Sciences, a standard reference text in this field.

Additionally, standard pharmaceutical methods can be employed to control the duration of action. These are well known in the art and include control release preparations and can include appropriate macromolecules, for example polymers, polyesters, polyaminoacids, polyvinylpyrrolidone, ethylenevinylacetate, methyl cellulose, caraboxymethyl cellulose or protamine sulfate. The concentration of macromolecules as well as a the methods of incorporation can be adjusted in order to control release. Additionally, the agent can be incorporated into particles of polymeric materials such as polyesters, polyaminoacids, hydrogels, poly (lactic acid) or ethylenevinylacetate copolymers. In addition to being incorporated, these agents can also be used to trap the compound in microcapsules.

Useful pharmaceutical dosage forms for administration of the compounds of this invention can be illustrated as follows.

Capsules: Capsules are prepared by filling standard two-piece hard gelatin capsulates each with 100 milligram of powdered active ingredient, 175 milligrams of lactose, 24 milligrams of talc and 6 milligrams magnesium stearate.

Soft Gelatin Capsules: A mixture of active ingredient in soybean oil is prepared and injected by means of a positive displacement pump into gelatin to form soft gelatin capsules containing 100 milligrams of the active ingredient. The capsules are then washed and dried.

Tablets: Tablets are prepared by conventional procedures so that the dosage unit is 100 milligrams of active ingredient. 0.2 milligrams of colloidal silicon dioxide, 5 milligrams of magnesium stearate, 275 milligrams of microcrystalline cellulose, 11 milligrams of cornstarch and 98.8 milligrams of lactose. Appropriate coatings may be applied to increase palatability or to delay absorption.

Injectable: A parenteral composition suitable for administration by injection is prepared by stirring 1.5% by weight of active ingredients in 10% by volume propylene glycol and water. The solution is made isotonic with sodium chloride and sterilized.

Suspension: An aqueous suspension is prepared for oral administration so that each 5 millimeters contain 100 milligrams of finely divided active ingredient, 200 milligrams of sodium carboxymethyl cellulose, 5 milligrams of sodium benzoate, 1.0 grams of sorbitol solution U.S.P. and 0.025 millimeters of vanillin.

The therapeutic compound of the present invention comprises at least one drug moiety, and at least one polypeptide drug carrier moiety, the drug moiety being covalently linked to the carrier moiety.

In the practice of the present invention, the therapeutic compound may comprise any suitable amounts of drug moiety(ies) and drug carrier moiety. Generally, the therapeutic compound comprises from about 10 percent to about 60 weight percent drug moiety, more preferably from about 20 percent to about 50 weight percent drug moiety, and most preferably from about 20 percent to about 40 weight percent drug moiety. Generally, the therapeutic compound may comprise from about 40 percent to about 90 weight percent drug carrier moiety, more preferably from about 50 percent to about 80 percent, weight percent drug carrier moiety, and most preferably from about 60 percent to about 80 percent, weight percent drug carrier moiety.

In the practice of the present invention, the molecular weight of the drug carrier moiety may be any suitable molecular weight that will accomplish the intended purpose of the compound, without substantial side effects. In general, this means that the molecular weight of the compound is in the range of about 10,000 daltons to about 100,000 daltons, preferably in the range of about 20,000 to about 50,000 daltons, more preferably in the range of about 26,000 to about 30,000 daltons.

The polypeptide drug carrier moiety of the present invention generally comprises glutamic acid and a second amino acid selected from the group consisting of aspartic acid, alanine, asparagine, glutamine, glycine, and combinations thereof. Preferably, the polypeptide drug carrier comprises repeating units of glutamic acid and aspartic acid such that when the carrier is covalently conjugated to at least one drug imparts enhanced solubility upon the drug(s) as well as enhanced and/or unique biological properties. This preferred embodiment is exemplified in examples below in the conjugation of paclitaxel to a polyglutamic acid-aspartic acid polypeptide and its use in, for example, treatment of prostate cancer.

In the present invention, while any suitable percentages of amino acids may be used, and may vary depending upon the drug moiety selected, the polypeptide drug carrier moiety may comprise glutamic acid in the range of about 50 to about 90 weight percent, preferably from greater than 50 to about 90 weight percent, more preferably from about 60 to about 80 weight percent, most preferably from about 70 to about 75 weight percent, with the second amino acid(s) making up the balance, that is, from about 10 to about 50 weight percent, preferably from 10 to less than 50 90 weight percent, more preferably from about 20 to about 40 weight percent, most preferably from about 25 to about 30 about 75 weight percent.

It is to be understood that the present invention is not limited to the conjugation of any particular drug but rather, encompasses the conjugation of a wide range of drugs, both known and presently unknown, readily water soluble or poorly water soluble and of varying biological effects. Thus, in the practice of the present invention, the drug moiety may be selected from the group consisting of anti-tumor drugs, anti-inflammatory drugs, drugs for the cardiovascular system, diabetic drugs, metabolically-acting drugs, drugs for pain treatment and any other types of drugs where delivery via the inventive carrier is or may be desired. Preferably, the drug moiety may be selected from the group of drugs consisting of paclitaxel, epipodophyllotoxin, podophyllotoxin, vincristine, docetaxel, daunomycin, doxorubicin, mitoxantrone, topotecan, bleomycin, gemcitabine, fludarabine and 5-FUDR. More preferably, the drug moiety is selected from the group consisting of paclitaxel, epiepdophyllotoxin, decetaxel, topotecan, and podophyllotoxin. Even more preferably, the drug moiety is paclitaxel.

In the present invention, the therapeutic compound may comprise at least two drug moieties, which may not the same as each other, and/or the therapeutic compound may comprise a plurality of drug moieties.

In the present invention, while the therapeutic compound and the drug moiety may have any suitable water solubilities that will provide the desired results, preferably, the water solubility of the therapeutic compound is greater than the water solubility of the drug moiety.

Conditions to be treated may include, but are in no way limited to, prostate, breast, ovarian, colon, leukemia, lymphoma, lung and liver cancers. For example and in a non-limiting sense, paclitaxel may be conjugated with the inventive peptide and used to treat, for example, prostate, breast, ovarian, colon, leukemia, lymphoma, lung and liver cancers. Preferably the condition to be treated with the present invention is prostate cancer.

In a preferred embodiment, the drug moiety may be paclitaxel, the carrier moiety may comprise about 70 percent glutamic acid and about 30 percent aspartic acid, the paclitaxel drug moiety may be about 20 percent to about 40 percent, by weight, of the therapeutic compound, and the molecular weight of the compound may be from about 20,000 to about 50,000 daltons.

Another aspect of the present invention relates to a method for improving the solubility of the drug moiety described above comprising the steps of covalently conjugating at least one drug moiety as described above with at least one polypeptide drug carrier moiety as described above.

Still another aspect of the present invention relates to a method for treating a condition comprising the steps of administering a therapeutically effective amount of the above described therapeutic compound.

Furthermore, it is not necessarily contemplated that the present invention be limited to strictly the use of a polypeptide containing repeating monomers comprised of aspartic acid and glutamic acid, or alanine and glutamic acid, or asparagine and glutamic acid, or glutamine and glutamic acid, or glycine and glutamic acid.

For example, a polyglutamic acid as follows:

NH₂—[-G-G-G-G-G-G-G-G-G-G-]ₙ—COOH

wherein G is glutamic acid.

While at this time, a preferred embodiment is a polymer consisting of glutamic acid and aspartic acid (and indeed details of the preferred embodiment are provided in the illustrative examples below), it is not contemplated that this be a limited example of the full scope of the invention. For example, it is contemplated that the inventive polymer drug carrier need not be exclusively composed of polyglutamic acid/aspartic acid (or alanine, or asparagine, or glutamine, or glycine) either as repeating monomer polypeptides or in mixed combinations. Indeed the noted amino acids (for example, glutamic acid and aspartic acid (or alanine, or asparagine, or glutamine, or glycine)) may make up some percentage of the overall polypeptide carrier. Further, the carrier may comprise other components than the noted amino acids, providing that at least some of the carrier is composed of the inventive polypeptide combination.

The present invention is not restricted solely to use of “wild type” amino acids in the polymer. Rather, the invention encompasses any number of changes to the structure of these amino acids which would result in polypeptides having essentially the same function and/or structure. The amino acids may be D amino acids, L amino acids or mixtures of D and L amino acids. Further still, it is contemplated that the drug conjugate peptide of the present invention need not exclusively contain an individual polypeptides containing 100% glutamic/aspartic acid (or alanine, or asparagine, or glutamine, or glycine). Rather, while sections of the polypeptide may contain the noted amino acids, it is believed that it is not necessary for the entire peptide to homogeneously include the only the noted amino acids, especially not necessarily in repeating monomers.

Optionally, any of the polypeptides of the present invention, may incorporate, include or be linked to or bonded to a “homing agent” which preferentially binds to a receptor on the target tumor cell. Specifically, such a homing agent is a non-immunoglobulin covalently linked to the polypeptide, and which preferentially binds to a receptor on the target tumor cell. However, preferably, the therapeutic compound of the present invention comprises a drug moiety, the polypeptide drug carrier moiety, and other than these moities which might function as “homing agents,” is free of any homing agent which preferentially binds to a receptor on the target tumor cell.

The polypeptide drug carrier moiety of the present invention may optionally be modified to provide local lipophilicity at one or both termini, but preferably lacks, that is free of, local lipophilicity at both termini, more preferably comprise unmodified termini.

In the practice of the present invention, it is preferred that the polypeptide drug carrier moiety may be a copolymer of two or more amino acids. It is noted that while U.S. Pat. No. 6,441,025 discloses that a “water soluable polyamino acid” may include “amino acid chains comprising mixtures of glutamic acid, aspartic acid, and/or lysine,”it does not disclose the configuration of such.

For example, a polyamino acid comprising glutamic acid and aspartic acid may have a block structure as follows:

NH₂—[-G-G-G-G-G-]ₘ—CO—NH—[-A-A-A-A-A-]ₙ—COOH

wherein G is glutamic acid, and A is aspartic acid. Note that nonoe of the glutamic acids is directly bonded to any of the apspartic acids.

However, a polyamino acid comprising glutamic acid and aspartic acid may also have an interspersed structure as follows:

NH₂—[-G-A-G-A-G-A-A-G-G-A-]ₙ—COOH

again, where G is glutamic acid and A is aspartic acid. Note that at least one of the G's is directly bonded to at leasta one of the A's

It is noted that only four types of combinations of glutamic with a second amino acid are possible. For example, illustrating with glutamic and aspartic, would provide, GAG, AGA, GA or AG.

The present invention prefers this structure, in which the various amino acids are interspersed among each other, either in random form, in one or more regular patterns, or in interspersed block forms (i.e., GGGGAAGGGGAAGGGGAA, etc).

Preferably, the polypeptide drug carrier moiety comprises glutamic acid and a second amino acid, with at least one of the glutamic acids bonded directly to at least one second amino acid, preferably bonded directly to two second amino acids. Preferably, at least 10% of the glutamic acids, more preferably at least 20%, even more preferably at least 50%, still more preferably at least 60%, and yet more preferably at least 75% of the glutamic acids are bonded directly to at least one second amino acid. Most preferably, the second amino acid is aspartic.

In the preferred glutamic acid/second amio acid polypeptide drug carrier moiety, preferably at least 10% of the drug moiety is directly bonded to glutamic acid, more preferably at least 50%, even more preferably at least 60%, still more preferably at least 75%, and yet more preferably at least 90%.

### EXAMPLES

The following detailed examples of the preferred embodiment relate to an illustrative example of the present invention wherein the poorly soluble antitumor drug paclitaxel is conjugated to the inventive di(glutamate/aspartate) polypeptide drug carrier and the resulting product is shown to have unique and indeed surprising biological activity, for example, against prostate cancer in vivo. Also disclosed are methods for producing the inventive polypeptide drug carrier and sample conjugates.

It is to be understood that these examples are in no way intended to limit the scope of the present invention but merely illustrate one example of a preferred embodiment presently known to the inventors. Additional, embodiments are within the scope of the present invention.

### Example 1

### Synthesis of poly(dipeptide) polypeptide

The following sections report on preferred, but not limiting, embodiments for synthesizing the inventive polyglutamic acid/aspartic acid (or poly glutamic acid/alanine, or poly glutamic acid/asparagine, or poly glutamic acid/glutamine, or poly glutamic acid/glycine) copeptide and properties of such a peptide. In general, the inventive poly(glutamic/aspartic acid) di-peptide is a biodegradable polymer. As described below, the polypeptide may be synthesized in a conjugate form with a particular drug in order to enhance the solubility and/or in vivo deliverability of such drug. In such an instance, it may be considered as a “propolymeric drug delivery vehicle” and be prepared in a powder form. By adding sterile to the powder, the drug conjugate can then be used for interveneous administration. The inventive polymer-drug conjugates provide sustained relief properties and prolong blood circulation time which are more effective and less toxic than using, for example, unconjugated drug alone. As discussed above, examples of drugs which can be conjugated to the inventive conjugating include, in a non-limiting sense, paclitaxel, epipodophyllotoxin, vincristine, docetaxel, daunomycin, doxorubicin, mitoxantrone, topotecan, bleomycin, gemcitabine, fludarabine and 5-FUDR

In one embodiment, for example, the inventive poly(glutamate/aspartate) polypeptide is approximately 26,000 to 30,000 dalton molecular weight containing approximately 70% glutamic acid and 30% aspartic acid. Hence, it can be seen that the inventive copolymer need not necessarily contain a homogeneous and repeating di-peptide, which would result in a 50-50 content of glutamic acid and aspartic acid. Rather, many variations within this range are contemplated. For example, the preferred embodiment may contain 70% glutamic acid and 30% aspartic acid. However, this range could extend from 50-90% (weight) glutamic acid and 10-50% (weight) aspartic acid.

### Example 2

### Experimental Procedure-Synthesis of Poly(glutamate/aspartate) Polypeptides

Using a known procedure, N-carboxyanhydride (NCAs) was prepared by phosgenation of the corresponding β-benzyl-1-aspartate and γ-benzyl-1-glutamate (Idelson, M., Blout, E. R., J. Am. Chem. Soc. 1958, 80, 2387-2393; Karlson, R. H., Norland, K. S., Fasman, G. D., Blout, E. R., J. Am. Chem. Soc. 1960, 82, 2268-2278; Paolillo, L., Temussi, P. A., Bradbury, E. M., Crane-Robinson, C. Biopolymers, 1972, 11, 2043-2052; Hayashi, T., Iwatsuki, M., Biopolymers, 1990,29,549-557; Bradbury, E. M., Carpenter, B. G., Crane-Robinson, C., Goldman, H.,Macromolecules, 1971,4,557-564.). Briefly, a solution of phosgene (10% w/v) was bubbled into ethylacetate (150 ml). An aliquot (10 ml) of this solution was added to 10 grams of finely ground β-benzyl-1-aspartate and γ-benzyl-1-glutamate in ethylacetate (150 ml). The reaction was stirred under reflux for 5 min. A stream of nitrogen was employed to remove excess HCI prior to the next addition of phosgene. The sequence was repeated until no traces of suspended amino acid HCl remained. The mixture was then filtered and the solvent was evaporated under Vacuo. The product was crystallized from ethyl acetate.

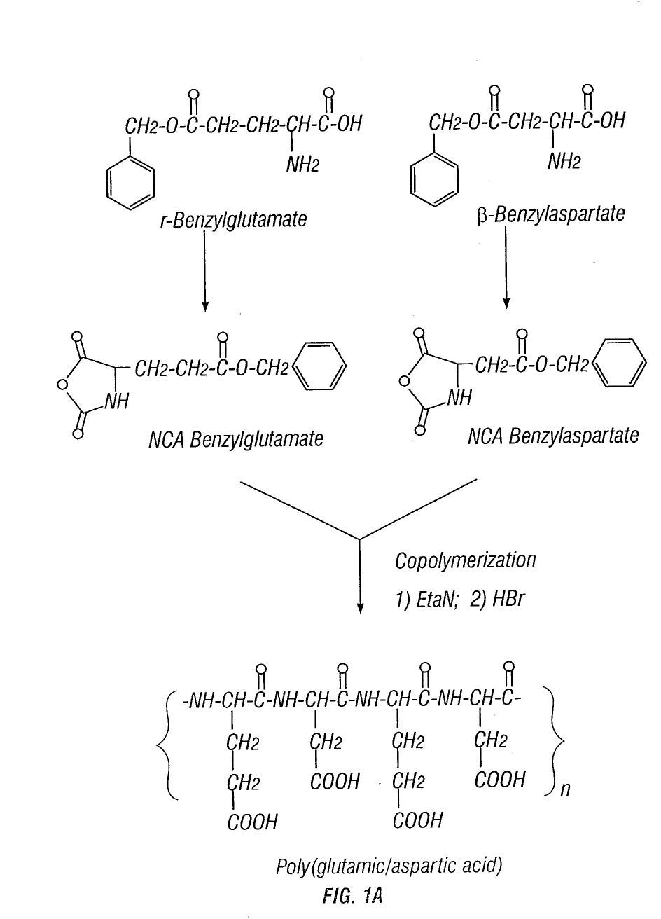

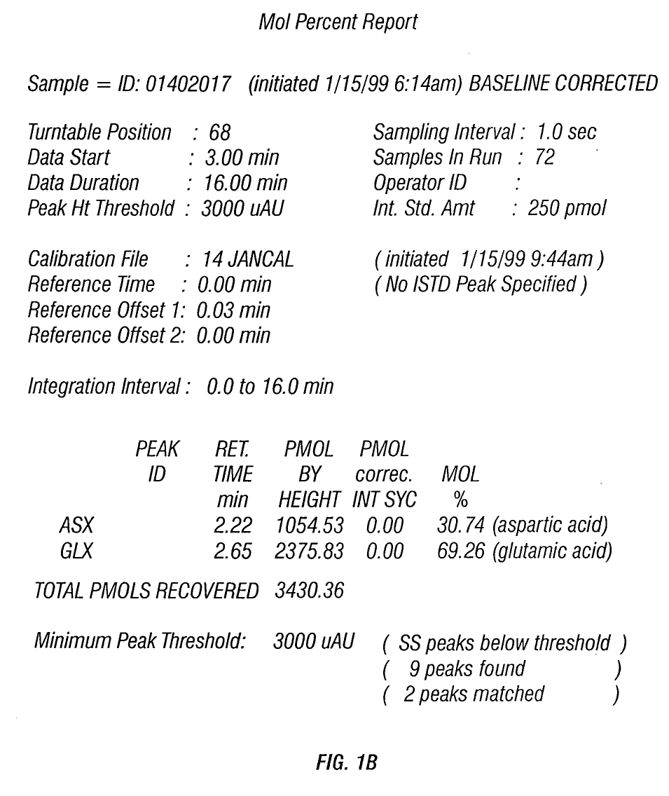

Solutions of NCAs of β-benzyl-1-aspartate and γ-benzyl-1-glutamate in dioxane/methylene chloride (1:3) were prepared. The ratios (w/w) used between δ-benzyl-1-aspartate and γ-benzyl-1-glutamate were 3:7, 2:8 and 1:9. The polymerization was initiated with triethylamine in methylene chloride (4 ml, 2.5% v/v). The copolymerization reaction was under reflux for 30 min and followed by CO₂ evolution. The reaction was stopped at about 30 mol % conversion. The polymers formed were precipitated by adding ice cold methanol containing 0.IN HCI (5%) v/v). The products were washed with methanol and dried under reduced pressure, yielded 8 gm (for 3:7 batch). The debenzylation was conducted by using HBr according to a known procedure (Idelson, M.; Blout, E. R., J. Am. Chem. Soc. 1958, 80,2387-2393). After HBr treatment, the aqueous solution was dialyzed against distilled water, filtered through Millipore filter and lyophilized. Typical average molecular weight was 26,000-30,000 daltons. A synthetic scheme is shown in FIG. 1A. A similar technique was used to prepare polymers of glutamic acid and alanine, glutamic acid and asparagine, glutamic acid and glutamine, glutamic acid and glycine, and glutamic acid and one or more amino acids from the group consisting of aspartic acid, alanine, asparagine, glutamine, and glycine.

Amino acid analyzer (PE/ABI 42OA) (Foster City, Calif.) was used to determine the actual composition ratio of aspartic acid and glutamic acid. Briefly, poly(dipeptide) was hydrolyzed with HCI (6N) at 150° C. for 75 min. The hydrolyzed products were loaded on PVDF membrane and methanol (30%) and HCI (0.1N, 0.2 ml) were added to extract the amino acids. Using pre-column derivatization with phenylisothiocyanate, the amino acid concentration was determined. An amino acid analysis of the poly(glutamic acid/aspartic acid) is shown in FIG. 1B.

### Example 3

### Anti-Cancer Drug Delivery Using Poly(glutamate/aspartate) Polypeptide as a Drug Carrier

In order to demonstrate one embodiment of the present invention, a conjugate of the antitumor agent paclitaxel was made with the inventive polypeptide and used as a drug delivery vehicle. It was then shown that this inventive conjugate possessed superior biological and therapeutic properties in vivo over, for example, unconjugated drug, and the drug conjugated with known prior art carriers (e.g., homopolymers of glutamic acid and aspartic acid). Data below shows that, for example, conjugating the antitumor drug paclitaxel to the inventive polymer—and only the inventive polymer—results in unexpected therapeutic properties of paclitaxel such as the treatment of prostate cancer. Indeed, the following results show what applicants believe is the first described efficacy of paclitaxel in any form against prostate tumors in vivo.

### Example 4

### Experimental Procedure-Synthesis of Poly(zlutamate/aspartate)—Paclitaxel Conjugates

Numerous studies have suggested that limited polymer-drug conjugate discretion through the kidneys is evident when the molecular weight of the conjugate ranges from 20,000 to 50,000 daltons. Thus, to enhance tumor uptake of the paclitaxel-inventive carrier conjugate, a molecular weight range of a conjugate of 26,000 to 30,000 daltons was selected.

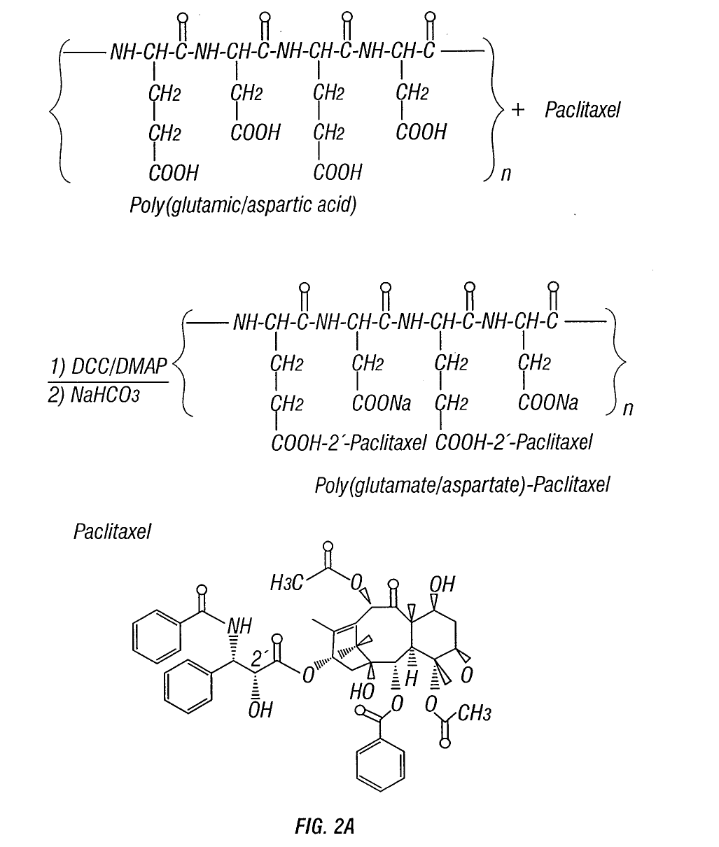

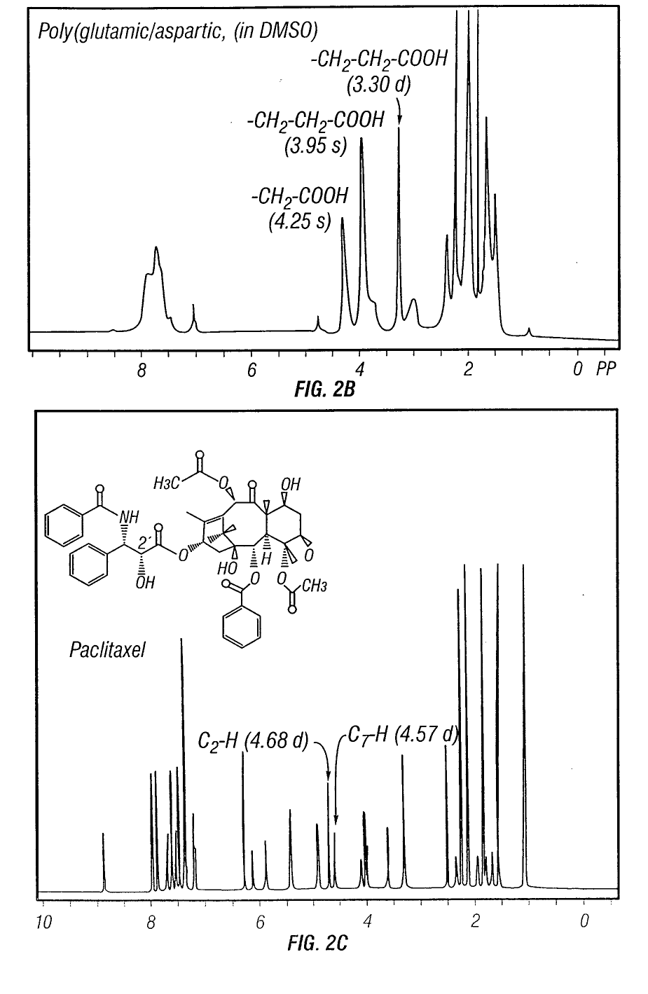

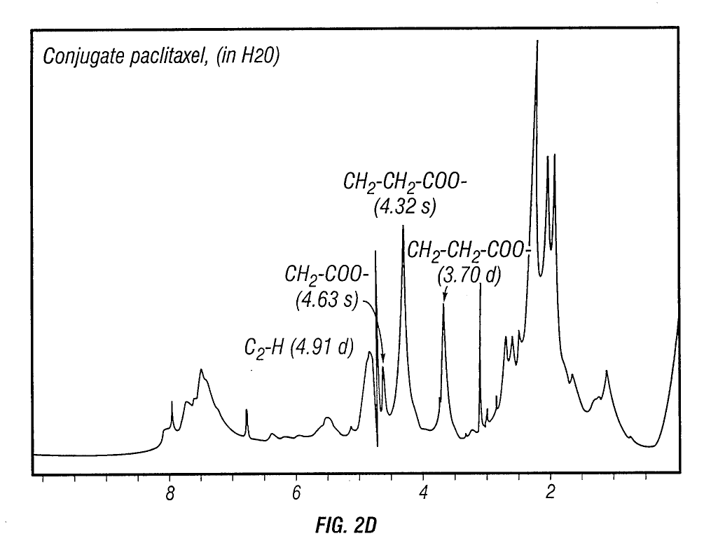

Conjugation of paclitaxel to poly(glutamate/aspartate) was conducted by using drug:polymer molar ratio of 1:4 in N,N-dimethylformaide (DMF). Dicyclocarbodiimide (DCC) was used as a coupling agent. In a typical run, poly(dipeptide) (383 mg) was dissolved in DMF (8 ml) and DCC (152.2 mg) was added. To this mixture, N,N-dimethylaminopyridine (8.5 mg) and paclitaxel (209.4 mg) were added. The reaction was stirred for 22 hours under room temperature. The urea was filtered and the resulting solvent was added to chloroform. The product was filtered and redissolved in sodium bicarbonate (IN). After dialysis (cut off at 10,000) against distilled water, the product was freeze-dried and weighed; 620 mg. The product contained 26.79% paclitaxel. Nuclear magnetic resonance (NMR) spectra was recorded on a GE GN-500 Spectrometer. A synthetic scheme of conjugating paclitaxel to poly(glutamate/aspartate) is shown in FIG. 2A.

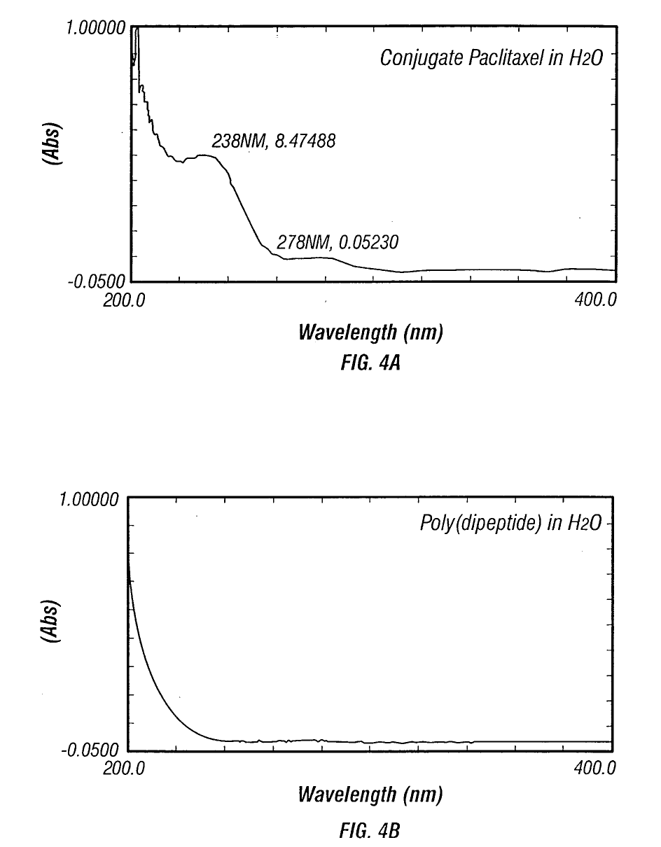

Proton nuclear magnetic resonance (¹HNMR) of poly(dipeptide), paclitaxel and water soluble poly(dipeptide)-paclitaxel conjugates were conducted using GE 600 MHz NMR. The spectrums are shown in FIGS. 2B, 2C and 2D. In the paclitaxel conjugates, apparently, C2′ position of paclitaxel was linked to the conjugates. For instance, the chemical shift (δ) of C2′ was 4.68 (doublet (d) in paclitaxel (FIG. 2B) and 4.91(d) in the paclitaxel conjugates (FIG. 2D).

### Example 5

### Determination of paclitaxel concentration in poly(glutamate/aspartate) paclitaxel conjugates:

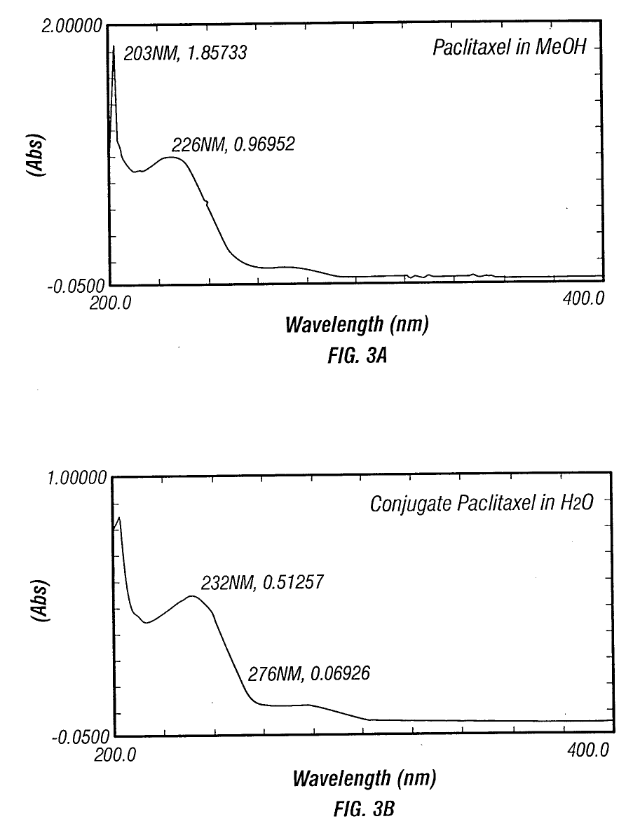

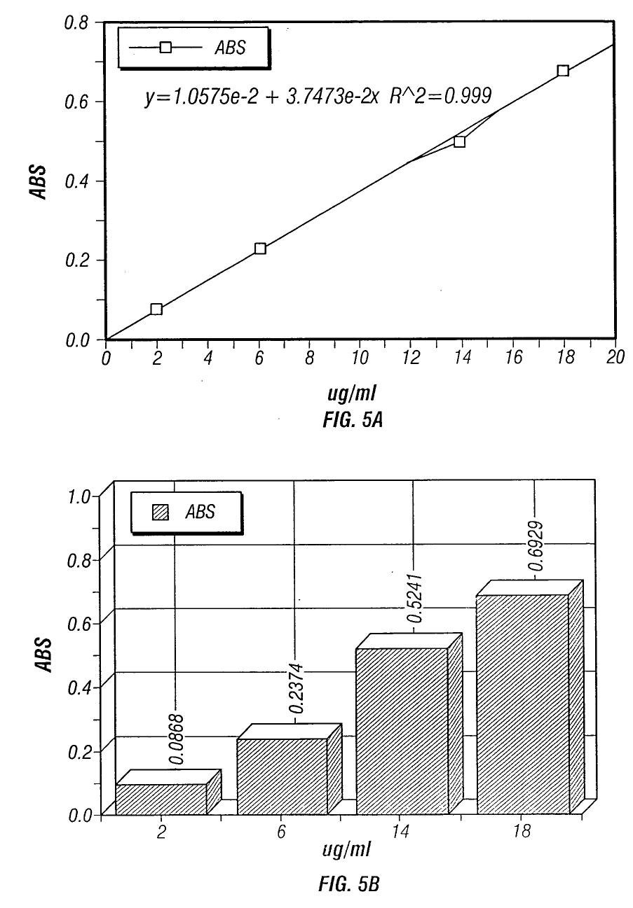

To compare the difference between paclitaxel and its conjugates in ultra violet (UV) absorption, UV scans (Beckman DU-640 spectrometry, Fullerton, Calif.) of these derivatives were recorded (FIGS. 3A, 3B, 4A and 4B). Absorbency of paclitaxel at various concentrations in methanol (2,6,14 and 18 μg/ml) was determined by UV at 232 nm. A standard curve was then generated (FIG. 5A). Polymer paclitaxel conjugates was dissolved in water and absorbency of an aliquot of this solution was determined. Paclitaxel concentration in the conjugates was determined by extrapolating to the standard curve (FIG. 5B).

### Example 6

### Chromatographic analysis of poly(zlutamate/aspartate) paclitaxel conjugates:

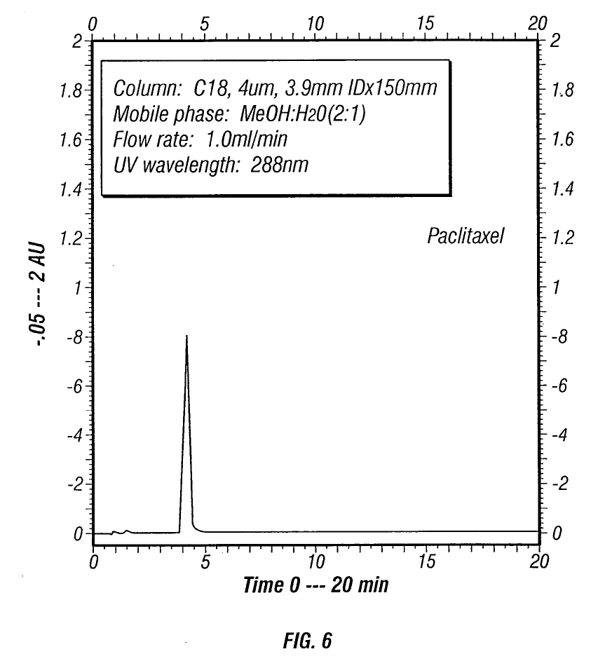

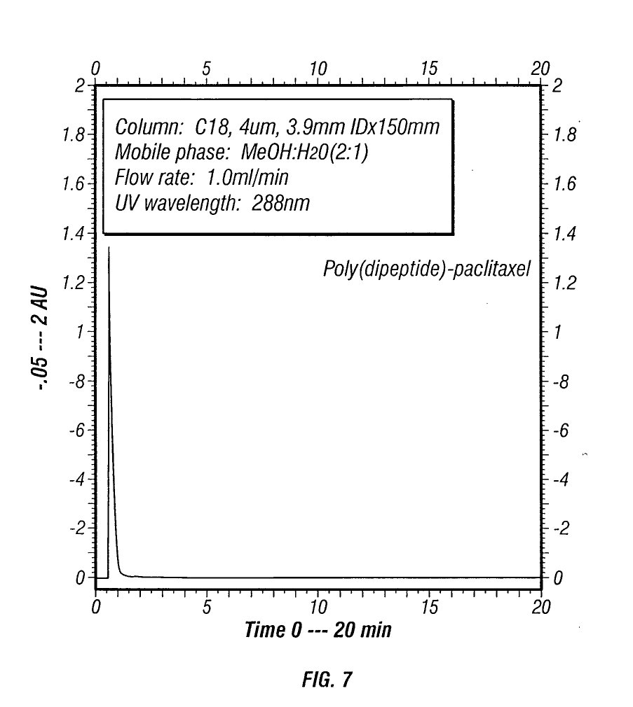

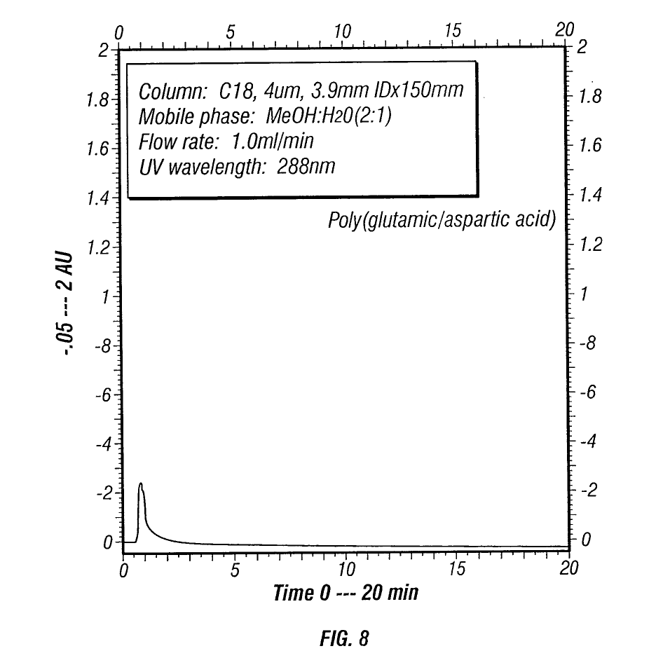

To demonstrate the purity of poly(glutamate/aspartate) paclitaxel conjugates, high performance liquid chromatography (HPLC) and thin-layer chromatography (TLC) were used. For HPLC, a Nova-Pak C-18 reverse phase column (3.9×15 mm) was used (FIGS. 6, 7, 8, 9A, 9B and 9C). The compounds were under the same concentration and eluted with methanol/water (2:1) at the flow rate of a ml/min. For TLC, a silica gel-coated plate was used. The product was eluted with chloroform/methanol (7:3).

### Example 7

### Solubility and stability of poly(glutamate/aspartate) paclitaxel conjugates:

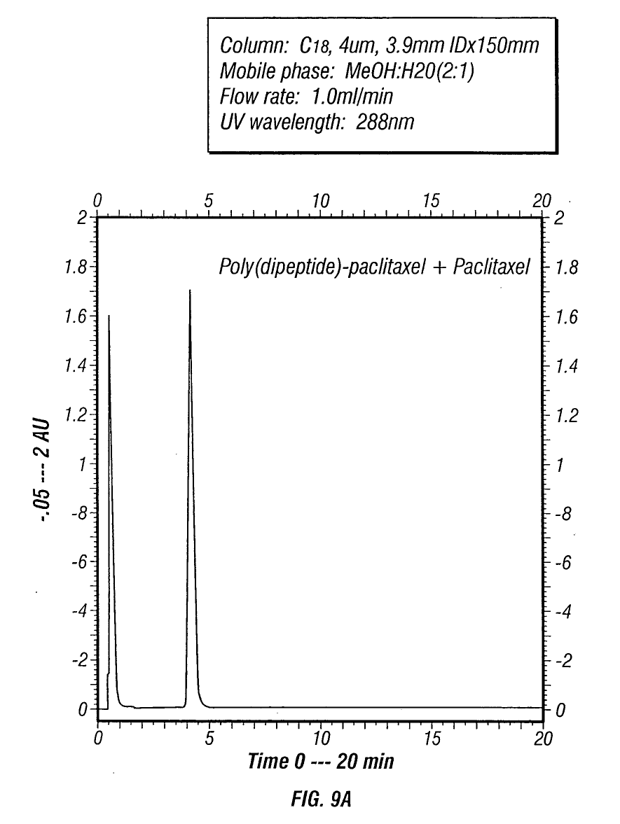

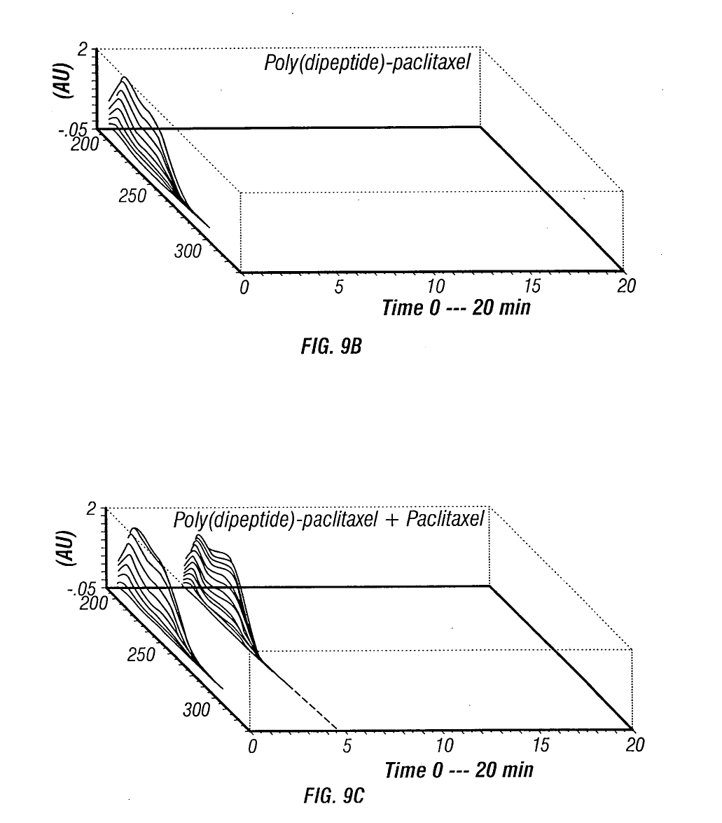

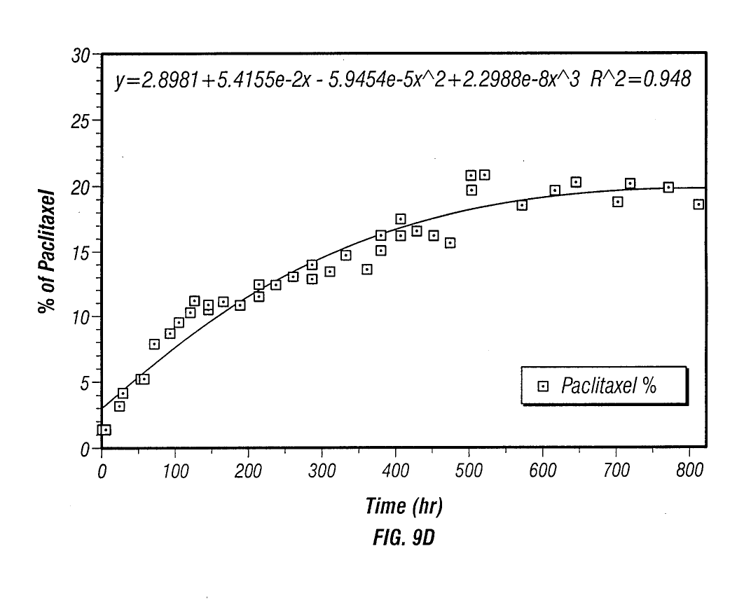

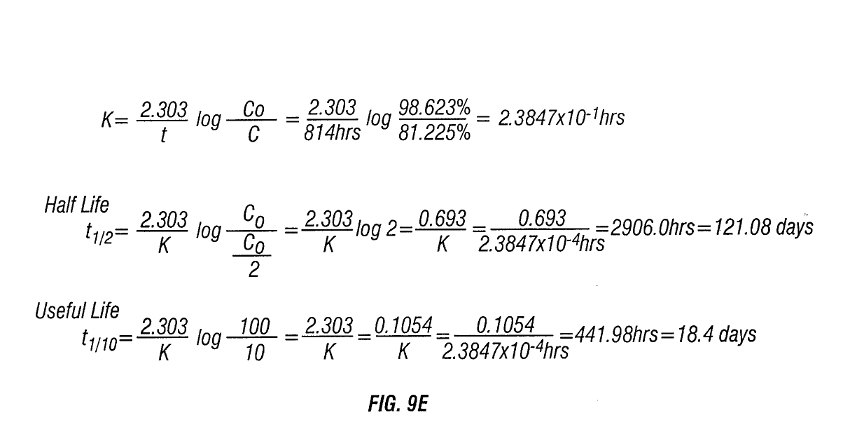

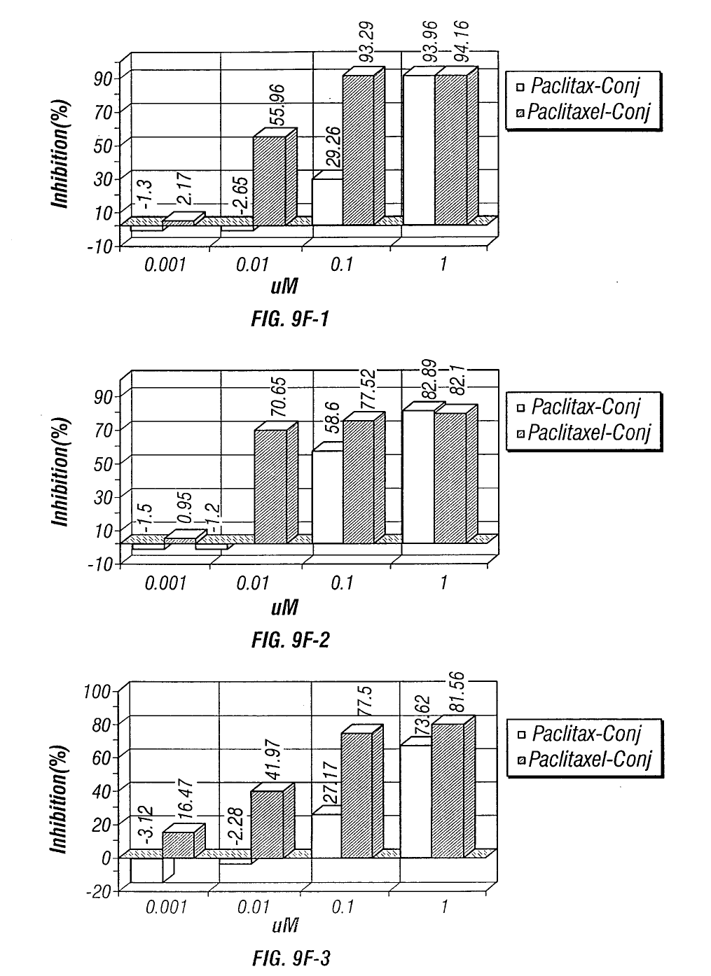

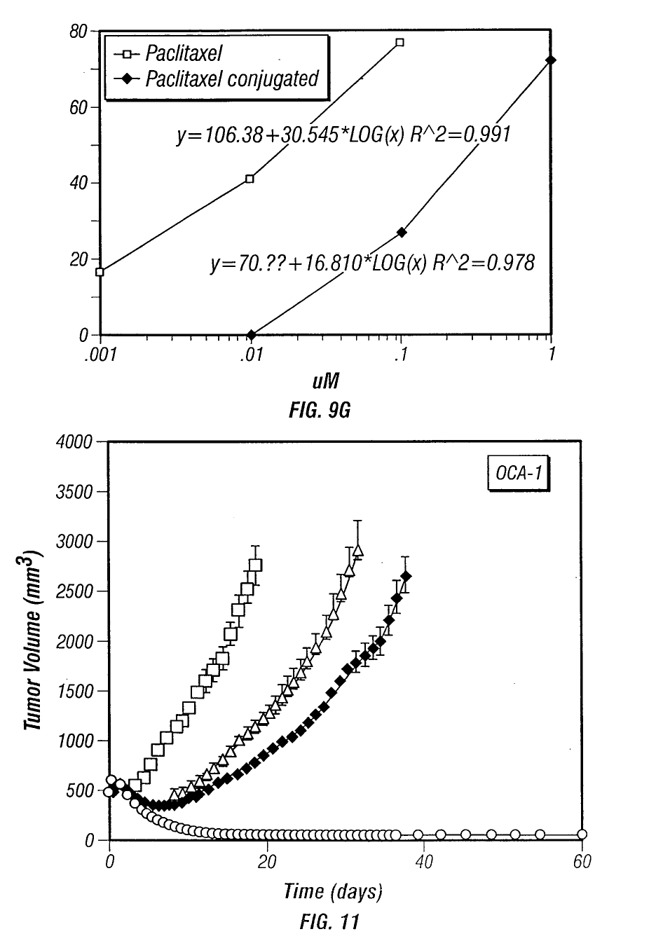

Solubility of the conjugates was determined in saline (0.9%) at 25° C. Stability assay was conducted in phosphate buffered saline (pH 7.4) at 25° C. An aliquot of sample at various time was assayed by HPLC (FIGS. 9D and 9E).

### Example 8

### In vitro cell culture assay:

To evaluate cytotoxicity of paclitaxel and the conjugates against mammary tumor cells, three human tumor cell lines were selected: PC3 (prostate); KB (nasopharyngeal); and, MDA MB 231 (breast). All cells were cultured at 37° C. in a humidified atmosphere containing 5% CO₂ in Eagle's medium. Forty-eight hours prior to the experiment, the cells were transferred to 35 mm culture dishes at 5×10⁵ cells per dish and grown to 80% confluence. Cultured human tumor cells in 35 mm dishes were incubated with either paclitaxel or conjugates at various concentrations. The incubation was stopped at 72 hours. Methylene tetrazolium (MTT) dye assay determined the amount of viable cells. Cellular protein content was determined by Lowry assay. The drug concentration that inhibits 50% of cell growth was then determined. At higher concentrations (e.g., 1 micro molar) there was no difference in cell inhibition between paclitaxel and the conjugates. However, cell inhibition was more pronounced in the paclitaxel group at lower concentrations (FIG. 9F).

### Example 9

### Evaluation of the conjugates in four tumor-bearing animal models:

Paclitaxel is known to produce an anticancer effect against breast and ovarian tumors, and not in the treatment of prostate cancer. Therefore, four animal models were selected: ovarian, breast and two prostate cancer models. The ovarian animal model was driven from animal tumor cell line, the other three models were created using human cell lines xenografted in nude mice.

### Example 10

### Ovarian tumor-bearing animal model:

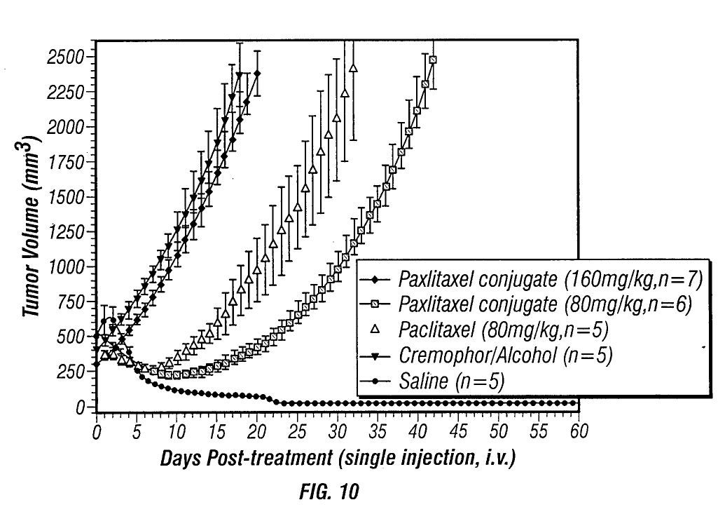

Female C3H/Kam mice (20-28 g, n=5/dose) were inoculated with ovarian tumor cells (OCA-1,500,000/mouse, subcutaneously (s.c.)) in the hind leg. When the tumor reached 500 mm³, the mice were administered either conjugates or paclitaxel doses of 40-160 mg/kg (conjugates) or 80 mg/kg (paclitaxel). For comparison purposes, a parallel study was conducted comparing our product with other water soluble paclitaxel products; mice were administered poly(glutamic acid) paclitaxel conjugates at doses of 40-160 mg/kg. Tumor volumes and body weight were recorded daily for sixty days. Tumor volumes were measured: [length (l)×width (w)×thickness (h)]/2. Loss of body weight of 15% is considered a chemical-induced toxic effect. These results are shown in FIGS. 10 and 11 and indicate that while conjugation of paclitaxel to a polypeptide enhances its anti-ovarian tumor efficacy substantially, there is no apparent difference between paclitaxel in the inventive conjugate compared with paclitaxel conjugataed to a prior art homopolymer carried polyglutamic acid.

### Example 11

### Breast tumor-bearing animal model:

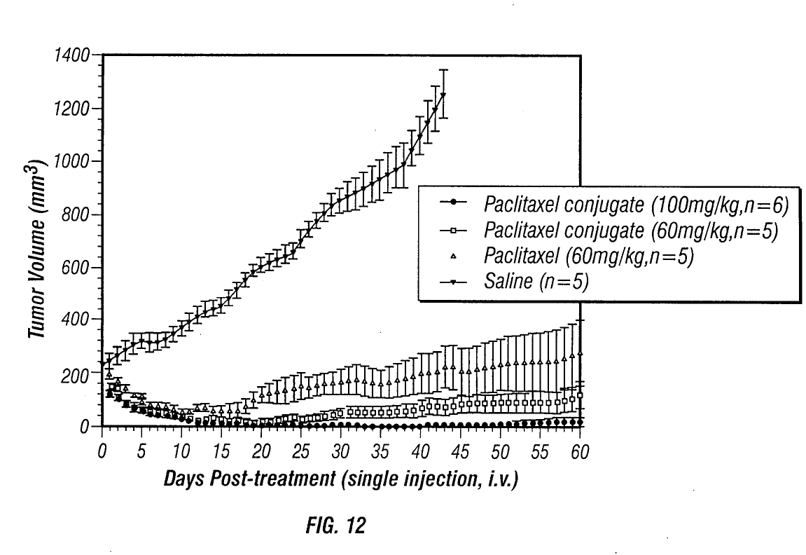

Athymic female nude mice (NCr5-nu/mu) were inoculated with human breast cancer cells (MDA435, 10⁶ cells/mouse, n=5/dose s.c. in the mouse mammary fat pad. After 15-20 days and a tumor volume of 250 mm³, the mice-bearing human breast tumor were administered either the conjugates or paclitaxel at doses of 60-100 mg/kg (conjugates) or 60 mg/kg (paclitaxel). Tumor volumes and body weight were recorded daily for sixty days. Tumor volumes were measured as [length (l)×width (w)×thickness (h)]/2. Loss of body weight of 15% is considering a chemical-induced toxic effect. The results are shown in FIG. 12 and indicate that unconjugated paclitaxel, paclitaxel conjugated to a prior art cancer polyglutamic acid humopolymer and the inventive conjugate are all effective in vivo against human breast cancer.

### Example 12

### Prostate tumor-bearing animal models:

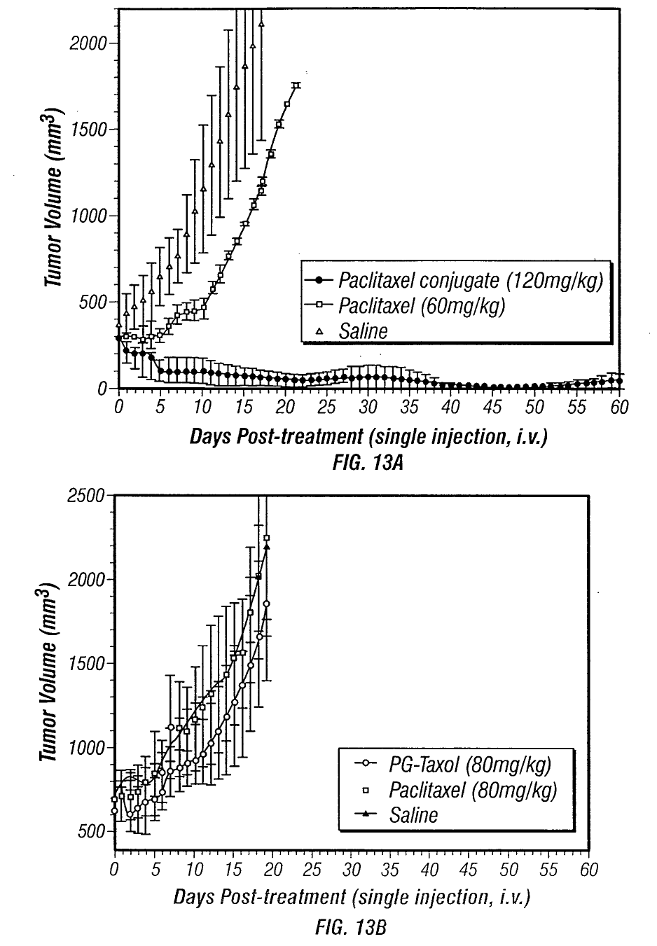

Athymic male nude mice (NCr-nu/nu) were inoculated with two types of human prostate cancer cells (A10 and PC3, 10⁶ cells/mouse, n=5/dose) s.c. in the mouse mammary fat pad. Both cell lines were obtained from the Department of GU oncology at The University of Texas M.D. Anderson Cancer Center. A10 cell line expresses PSA and androgen receptors (22). PC3 cell line does not overexpress PSA and androgen receptors. After 15-20 days with tumor volume 500 mm³, the mice-bearing human prostate tumors were administered either the conjugates or paclitaxel at the doses 60-120 mg/kg (conjugates) or 60 mg/kg (paclitaxel). FIG. 13A shows that paclitaxel conjugated with polyglytamic acid/aspartic acid is effective against prostate cancer in vivo whereas unconjugated paclitaxel is not effective.

For comparison purposes, a parallel study was conducted comparing the inventive paclitaxel conjugate with other water-soluble paclitaxel products, including paclitaxel conjugated to the prior art polymer polyglutamic acid. Mice were administered with poly(glutamic acid) paclitaxel conjugates at the dose 120 mg/kg. Poly(glutamic acid) paclitaxel conjugates were prepared using a known procedure (Li, C.; Yu, D-F.; Newman, R. S.; Cabral, F.; Stephens L. C.; Hunger, N.; Milas, L.; Wallace, S. Cancer Res. 1998, 58,2404-2409). Tumor volumes and body weight were recorded daily up to sixty days. Tumor volumes were measured as [length (l)×width (w)×thickness (h)]/2. Loss of body weight of 15% is considered a chemical-induced toxic effect. FIG. 13B shows that paclitaxel conjugated to polyglutamic polypeptide is ineffective against prostate cancer in vivo.

### Example 13

### Histopathology of tumor tissue after treatment:

After treatment with either paclitaxel or the polymer conjugates, tumor tissues (breast and prostate) were dissected and embedded in formalin. The tumor tissue was fixed in paraffin and stained with eosin or hematoxycilin. Apoptosis process produced by paclitaxel or the polymer conjugates was recorded by microscopic observation.

### Example 14

### Synthesis of poly(glutamate/aspartate) conjugates of paclitaxel:

Poly(glutamate/aspartate) at 26,000-30,000 molecular weight range was prepared. The poly(dipeptide) contains 70% glutamic acid and 30% aspartic acid as determined from amino acid analyzer (FIG. 1C). Conjugation of paclitaxel to poly(glutamate/aspartate) was conducted by using drug:polymer molar ratio of 1:4 in N,N-dimethylformaide. UV scans of paclitaxel, the conjugates and poly(dipeptide) are shown in FIGS. 3 and 4. The standard curve is shown in FIG. 5. Typical paclitaxel concentration in conjugates ranged from 20-40%.

### Example 15

### Chromatographic analysis of poly(glutamate/aspartate) paclitaxel conjugates:

HPLC analysis of the product showed that retention times for paclitaxel, the conjugates and the polymer of 4.2 min, 1.0 min and 1.0 min as shown in FIGS. 6-8. Though no difference between the conjugates and the polymer was reflected, the absorbency of the conjugates was significantly higher than the polymer alone. When mixed, the known amount of paclitaxel, a different retention time was noted (shown in FIGS. 9A, 9B and 9C). For TLC, the retarded factor (Rf) for paclitaxel and the conjugates was 0.8 and 0.1.

### Example 16

### Solubility and stability of poly(glutamate/aspartate) paclitaxel conjugates:

Solubility of conjugates was determined to be 20 mg/ml in saline, which is almost 3,000 times better than paclitaxel. Stability assay showed the useful half-life of the conjugates was 18 days in phosphate buffered saline (pH 7.4) at 25° C. (FIGS. 9D and 9E).

### Example 17

### In vitro cell culture assay:

The cytotoxicity (IC-50) of paclitaxel is about twenty times more potent than the conjugates in the PC3 cell line tested (FIG. 9G). This difference may be due to sustained release of paclitaxel from the conjugates.

### Example 18

### In vivo antitumor activity studies:

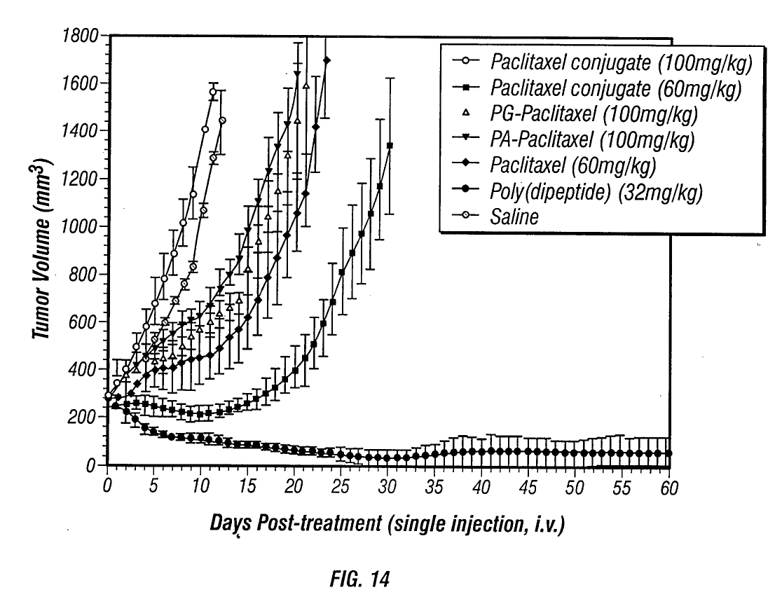

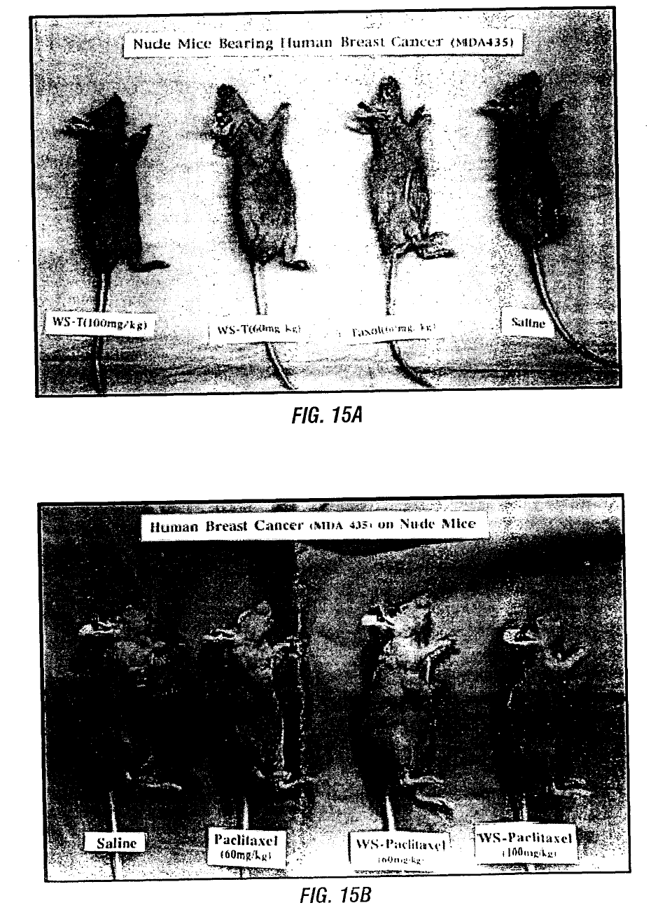

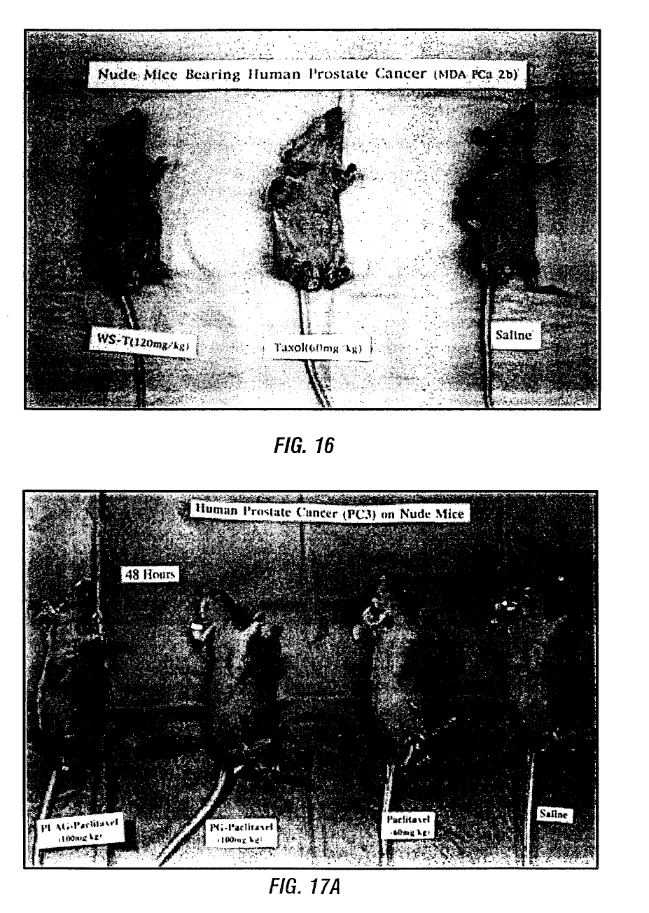

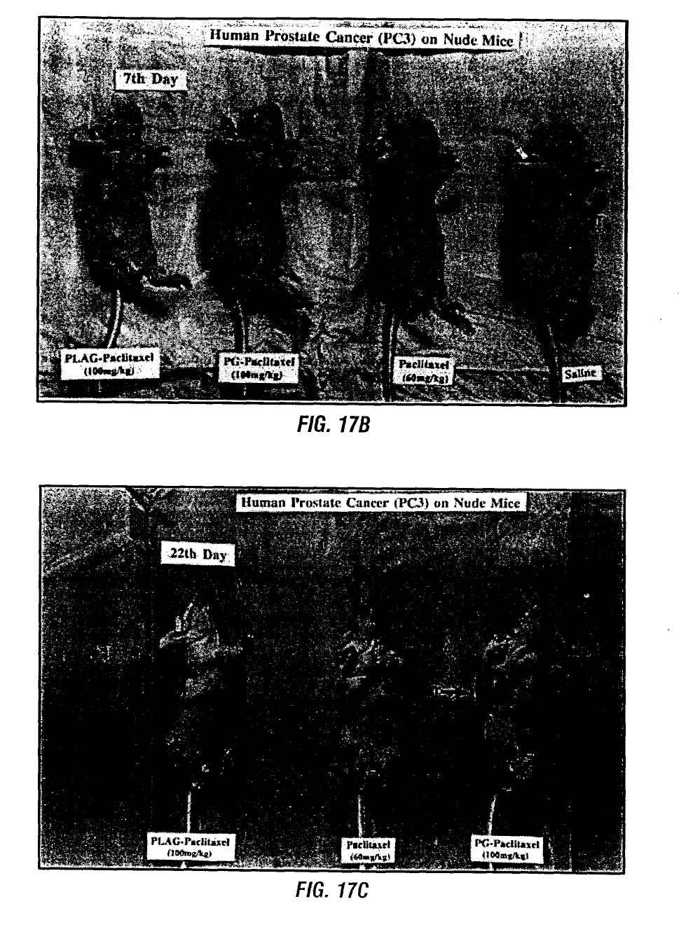

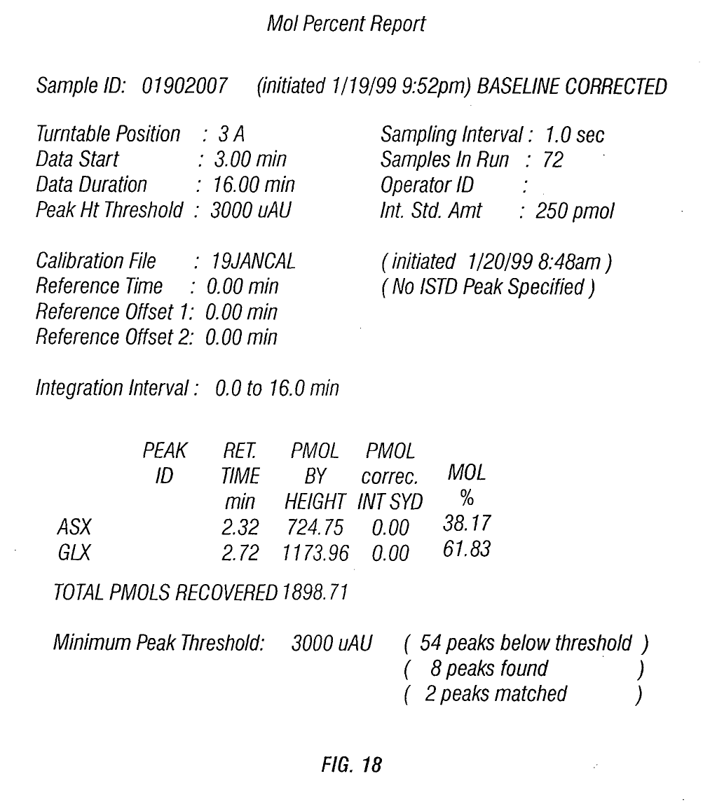

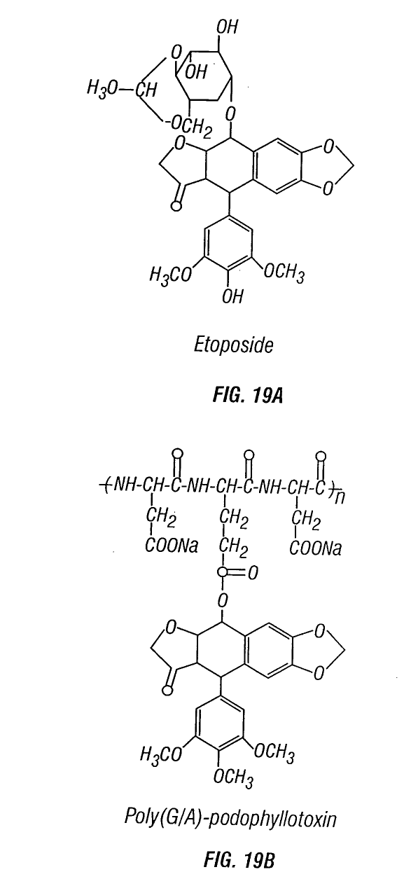

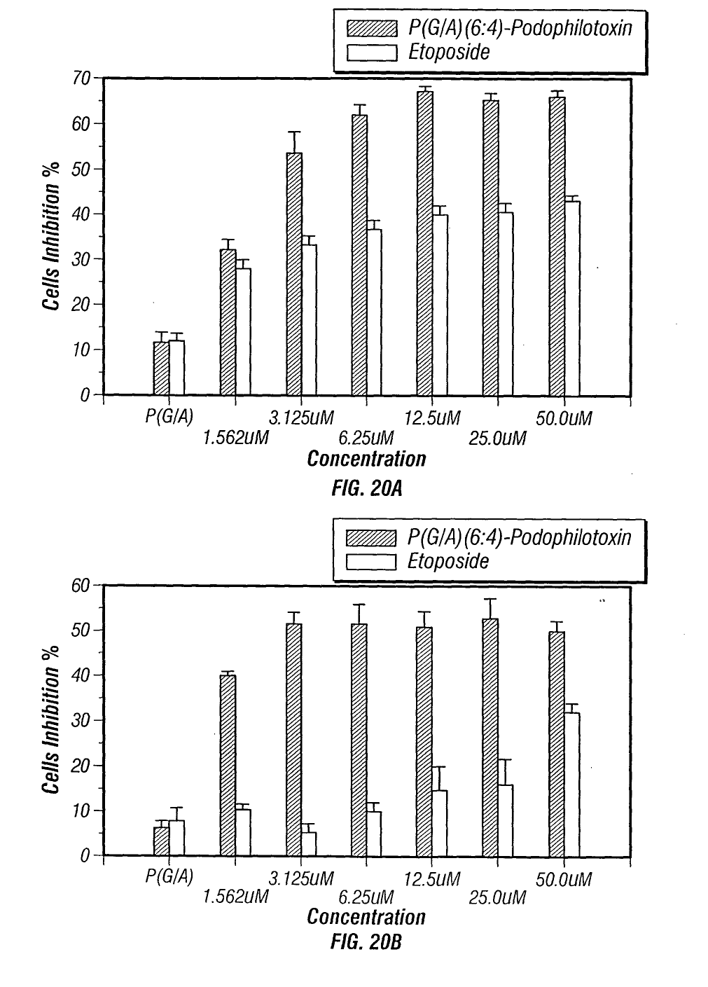

Poly(glutamate/aspartate) paclitaxel conjugates produced better antitumor effects than paclitaxel in all four animal models tested (shown in FIGS. 10, 12-14). When compared to poly(glutamic acid) paclitaxel conjugates, ply(glutamate/aspartate) paclitaxel conjugates had equivalent anti-tumor effects (FIGS. 10 and 11). However, poly(glutamate/aspartate) paclitaxel conjugates proved to be more potent than either poly(glutamic acid) paclitaxel or paclitaxel alone in prostate cancer animal models (shown in FIGS. 13A, 13B and 14). Despite the fact the higher initial loading dose could be provided by poly(glutamate/aspartate) paclitaxel conjugates, moreover, poly(glutamate/aspartate) paclitaxel conjugates did not alter body weight loss suggesting it is less toxic than paclitaxel. In FIGS. 15, 15B, 16, 17, 17B and 17C, tumor shrinkage in human tumor-bearing animal models for poly(glutamate/aspartate) paclitaxel conjugates is more pronounced than either poly(glutamic acid) paclitaxel or paclitaxel alone.

### Example 19

### Histopathology of tumor tissue after treatment:

After treatment with either paclitaxel or the polymer conjugates, apoptosis process produced by the polymer conjugates was more pronounced than paclitaxel using microscopic observation.

### Example 20

### Synthesis of Poly(glutamate/aspartate) Polypeptides

Using a known procedure, N-carboxyanhydride (NCAs) was prepared by phosgenation of the corresponding benzyl-1-aspartate and benzyl-1-glutamate (Idelson, M., Blout, E. R., J. Am. Chem. Soc. 1958, 80, 2387-2393; Karlson, R. H., Norland, K. S., Fasman, G. D., Blout, E. R., J. Am. Chem. Soc. 1960, 82, 2268-2278; Paolillo, L., Temussi, P. A., Bradbury, E. M., Crane-Robinson, C. Biopolymers, 1972,11, 2043-2052; Hayashi, T., Iwatsuki, M., Biopolymers, 1990,29,549-557; Bradbury, E. M., Carpenter, B. G., Crane-Robinson, C., Goldman, H.,Macromolecules, 1971,4,557-564.). Briefly, a solution of phosgene (10% w/v) was bubbled into ethylacetate (150 ml). An aliquot (10 ml) of this solution was added to 10 grams of finely ground benzyl-1-aspartate and benzyl-1-glutamate in ethylacetate (150 ml). The reaction was stirred under reflux for 5 mm. A stream of nitrogen was employed to remove excess HCI prior to the next addition of phosgene. The sequence was repeated until no traces of suspended amino acid HCI remained. The mixture was then filtered and the solvent was evaporated in vacuo. The product was crystallized from ethyl acetate.

Solutions of NCAs of benzyl-1-aspartate and y-benzyl-1-glutamate in dioxane/methylene chloride (1:3) were prepared. The ratios (w/w) used between benzyl-1-aspartate and benzyl-1-glutamate were 3:7, 2:8 and 1:9. The polymerization was initiated with triethylamine in methylene chloride (4 ml, 2.5% v/v). The copolymerization reaction was under reflux for 30 min and followed by CO₂ evolution. The reaction was stopped at about 30 mol % conversion. The polymers formed were precipitated by adding ice cold methanol containing 0.IN HCI (5%) v/v). The products were washed with methanol and dried under reduced pressure, yielded 8 gm (for 3:7 batch). The debenzylation was conducted by using HBr according to a known procedure (Idelson, M.; Blout, E. R., J. Am. Chem. Soc. 1958, 80, 2387-2393). After HBr treatment, the aqueous solution was dialyzed against distilled water, filtered through Millipore filter and lyophilized. Typical average molecular weight was 26,000-30,000 daltons. A synthetic scheme is shown in FIG. 1A. A similar technique was used to prepare polymers of glutamic acid and alanine, glutamic acid and asparagine, glutarnic acid and glutamine, glutamic acid and glycine, and glutamic acid and one or more amino acids from the group consisting of aspartic acid, alanine, asparagine, glutamine, and glycine. Amino add analyzer (PE/ABI 420 A) (Foster City, Calif.) was used to determine the actual composition ratio of aspartic acid and glutamic acid. Briefly, poly(dipeptide) was hydrolyzed with HCI (6N) at 150 for 75 min. The hydrolyzed products were loaded on PVDF membrane and methanol (30%) and HCI (0.IN, 0.2 ml) were added to extract the amino acids. Using pre-column derivatization with phenylisothiocyanate, the amino acid concentration was determined. An amino acid analysis of the poly(glutamic acid/aspartic acid) is shown in FIG. 1.

### Example 21

### Synthesis of Poly(glutamate/aspartate)—podophyllotoxin conjugates

Numerous studies have suggested that limited polymer-drug conjugate discretion through the kidneys is evident when the molecular weight of the conjugate ranges from 20,000 to 50,000 daltons. Thus, to enhance tumor uptake of the platinum analogue-inventive carrier conjugate, a molecular weight range of a conjugate of 26,000 to 30,000 daltons was selected.

Conjugation of podophyllotoxin to poly(glutamate/aspartate) was conducted as using podophyllotoxin:poly(dipeptide; ratio 6:4) molar ratio of 1:3 in N,N-dimethylsulfoxide (DMSO). Dicyclocarbodiimide (DCC) was used as a coupling agent. In a typical run, poly(dipeptide) (383 mg) was dissolved in DMSO (8 ml) and DCC (152.2 mg) was added. To this mixture, N,N-dimethylaminopyridine (8.5 mg) and podophyllotoxin (100 mg) were added. The reaction was stirred for 24 hours under room temperature. The urea was filtered and the resulting solvent was added to chloroform. The product was filtered and redissolved in sodium bicarbonate (IN). After dialysis (cut off at 10,000) against distilled water, the product was freeze-dried and weighed; 420 mg. The product contained 30% podophyllotoxin determined by UV analysis.

### Example 22

### In vitro cell culture assay:

To evaluate cytotoxicity of poly(dipeptide)-podophyllotoxin conjugate against mammary tumor cells, two human tumor cell lines were selected: LN-CAP (prostate); and, MDA 435 (breast). All cells were cultured at 37 in a humidified atmosphere containing 5% CO₂ in Eagle's medium. Forty-eight hours prior to the experiment, the cells were transferred to 35 mm culture dishes at 5×10⁵ cells per dish and grown to 80% confluence. Cultured human tumor cells in 35 mm dishes were incubated with either etoposide or poly(dipeptide)-podophyllotoxin conjugate at various concentrations. The incubation was stopped at 72 hours. Methylene tetrazolium (MTT) dye assay determined the amount of viable cells. Cellular protein content was determined by Lowry assay. The drug concentration that inhibits 50% of cell growth was then determined. The structures of etoposide and podophyllotoxin is shown in FIG. 2. An illustrated cell inhibition studies is shown in FIGS. 3 and 4. poly(dipeptide)-podophyllotoxin appears to be more active against tumor cells than etoposide.

In summary, a new poly(dipeptide) based water soluble paclitaxel is developed. The solubility is increased up to 20 mg/ml. The half-life of in vitro stability in phosphate buffered saline (pH 7.4) is 18 days. The product is easily scaled up and prepared as a sterilized powder. Compared to paclitaxel, insignificant toxicity was observed and much higher initial loading does could be administered intravenously. The product produced significant anticancer effects in ovarian, breast and prostate cancer models. In human prostate tumor-bearing nude mice, the product is effective as opposed to poly(glutamic acid) paclitaxel conjugates and unconjugated paclitaxel which did not show therapeutic effect against prostate tumors.

All patents and publications mentioned in this specification are indicative of levels of those skilled in the art to which the invention pertains. All patents and publications are herein incorporated by reference to the same extent as if each individual publication is specifically and individually indicated to be incorporated by reference.

One skilled in the art will readily appreciate the present invention is well adapted to carry out the objects and obtain the ends and advantages mentioned, as well as those inherent therein. The methods, procedures, treatments, molecules and specific compounds described herein are presently representative of preferred embodiments, are exemplary and are not intended as limitations on the scope of the invention. Changes therein and other uses will occur to those skilled in the art which are encompassed within the spirit of the invention and are defined by the scope of the claims.

What is claimed is:

1. A therapeutic compound comprising:
a drug moiety comprising paclitaxel,
at least one polypeptide drug carrier moiety having 70% by total weight of the polypeptide drug carrier, glutamic acid, and 30% by total weight by total weight of the polypeptide drug carrier, aspartic acid, wherein at least one glutamic acid is directly bonded to aspartic acid, and
the drug moiety being covalently linked to the carrier moiety.

2. The therapeutic compound of claim 1, wherein the drug carrier moiety comprises a molecular weight in the range of about 20,000 daltons to about 50,000 daltons.

3. The therapeutic compound of claim 1, wherein the drug moiety comprises from about 10 percent to about 60 percent, by weight, of the therapeutic compound.

4. The therapeutic compound of claim 1, wherein the drug moiety comprises from about 20 percent to about 50 percent, by weight, of the therapeutic compound.

5. The therapeutic compound of claim 1, wherein the drug moiety comprises from about 20 percent to about 40 percent, of the therapeutic compound.

6. The therapeutic compound of claim 1, wherein the amino acids can be in L form, or D form, or a racemic mixture of L and D forms.

7. The therapeutic compound of claim 1, wherein
the drug moiety comprises paclitaxel and is about 24 percent to about 30 percent, by weight, of the therapeutic compound, and
the molecular weight of the therapeutic compound is from about 26,000 to about 30,000 daltons.

8. A method for improving the solubility of a drug moiety comprising the steps of:
covalently conjugating the drug moiety with at least one polypeptide drug carrier moiety, thereby creating a therapeutic compound, the therapeutic compound comprising:
the drug moiety comprising paclitaxel, and
at least one polypeptide drug carrier moiety having 70% by total weight of the polypeptide drug carrier, glutamic acid, and 30% by total weight by total weight of the polypeptide drug carrier, aspartic acid, wherein at least one glutamic acid is directly bonded to at least one aspartic acid, and
the drug moiety being covalently linked to the carrier moiety.

9. The method of claim 8, wherein the drug carrier moiety comprises a molecular weight in the range of about 20,000 daltons to about 50,000 daltons.

10. The method of claim 8, wherein the water solubility of the therapeutic compound is greater than the water solubility of the drug moiety.

11. The method of claim 8, wherein
the drug moiety comprises paclitaxel and is about 24 percent to about 30 percent, by weight, of the therapeutic compound, and
the molecular weight of the therapeutic compound is from about 26,000 to about 30,000 daltons.

12. A method for treating a condition comprising the steps of:
administering a therapeutically effective amount of a therapeutic compound comprising:
a drug moiety comprising paclitaxel, and
at least one polypeptide drug carrier moiety having 70% by total weight of the polypeptide drug carrier, glutamic acid, and 30% by total weight by total weight of the polypeptide drug carrier, aspartic acid, and wherein at least one glutamic acid is directly bonded to at least one aspartic acid, and
the drug moiety being covalently linked to the carrier moiety.

13. The method of claim 12, wherein the drug carrier moiety comprises a molecular weight in the range of about 20,000 daltons to about 50,000 daltons.

14. The method of claim 12, wherein the condition is a prostate tumor.

15. The method of claim 12, wherein
the drug moiety comprises paclitaxel and is about 24 percent to about 30 percent, by weight, of the therapeutic compound, and
the molecular weight of the therapeutic compound is from about 26,000 to about 30,000 daltons.
